SEQUENCE LISTING (46 sequences)

SEQ ID NO: 1 - DNA, 20 bp, synthetic construct
  source 1..20: mol_type = other DNA
ctgacggacaacgggtctac

SEQ ID NO: 2 - DNA, 19 bp, synthetic construct
  source 1..19: mol_type = other DNA
gggtggcagaggaggaagt

SEQ ID NO: 3 - DNA, 40 bp, synthetic construct
  source 1..40: mol_type = other DNA
  modified_base 1..40: mod_base = OTHER; note = residues comprise a phosphorothioate internucleotide linkage
  modified_base 1..10: mod_base = OTHER; note = sugar group is modified with a 2 prime OMe
  modified_base 11: mod_base = OTHER; note = sugar group is a ribose
  modified_base 12..20: mod_base = OTHER; note = sugar group is modified with a 2 prime OMe
  modified_base 21: mod_base = OTHER; note = sugar group is a ribose
  modified_base 22..30: mod_base = OTHER; note = sugar group is modified with a 2 prime OMe
  modified_base 31: mod_base = OTHER; note = sugar group is a ribose
  modified_base 32..40: mod_base = OTHER; note = sugar group is modified with a 2 prime OMe
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = m5c
  modified_base 40: mod_base = m5c
acacacacacacacacacacacacacacacacacacacac

SEQ ID NO: 4 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 19..21: mod_base = OTHER; note = sugar group is modified with a 2 prime OMe
  modified_base 23: mod_base = OTHER; note = sugar group is a ribose
  modified_base 2: mod_base = m5c; mod_base = OTHER; note = residue is locked nucleic acid
  modified_base 4: mod_base = m5c; mod_base = OTHER; note = residue is locked nucleic acid
  modified_base 6: mod_base = OTHER; mod_base = m5c; note = residue is locked nucleic acid
  modified_base 8: mod_base = OTHER; mod_base = m5c; note = residue is locked nucleic acid
  modified_base 10: mod_base = m5c; mod_base = OTHER; note = residue is locked nucleic acid
  modified_base 12: mod_base = m5c; mod_base = OTHER; note = residue is locked nucleic acid
  modified_base 14: mod_base = m5c; mod_base = OTHER; note = residue is locked nucleic acid
  modified_base 16: mod_base = OTHER; mod_base = m5c; note = residue is locked nucleic acid
  modified_base 18: mod_base = OTHER; mod_base = m5c; note = residue is locked nucleic acid
  modified_base 20: mod_base = m5c
  modified_base 22: mod_base = OTHER; mod_base = m5c; note = residue is locked nucleic acid
  modified_base 24: mod_base = m5c; mod_base = OTHER; note = residue is locked nucleic acid
  modified_base 26: mod_base = m5c; mod_base = OTHER; note = residue is locked nucleic acid
  modified_base 28: mod_base = OTHER; mod_base = m5c; note = residue is locked nucleic acid
  modified_base 30: mod_base = OTHER; mod_base = m5c; note = residue is locked nucleic acid
  modified_base 32: mod_base = OTHER; mod_base = m5c; note = residue is locked nucleic acid
  modified_base 34: mod_base = m5c; mod_base = OTHER; note = residue is locked nucleic acid
  modified_base 36: mod_base = OTHER; mod_base = m5c; note = residue is locked nucleic acid
  modified_base 38: mod_base = m5c; mod_base = OTHER; note = residue is locked nucleic acid
  modified_base 40: mod_base = m5c; mod_base = OTHER; note = residue is locked nucleic acid
  modified_base 3: mod_base = OTHER; note = sugar group is modified with a 2 prime OMe
  modified_base 5: mod_base = OTHER; note = sugar group is modified with a 2 prime OMe
  modified_base 7: mod_base = OTHER; note = sugar group is modified with a 2 prime OMe
  modified_base 9: mod_base = OTHER; note = sugar group is modified with a 2 prime OMe
  modified_base 13: mod_base = OTHER; note = sugar group is modified with a 2 prime OMe
  modified_base 15: mod_base = OTHER; note = sugar group is modified with a 2 prime OMe
  modified_base 25: mod_base = OTHER; note = sugar group is modified with a 2 prime OMe
  modified_base 27: mod_base = OTHER; note = sugar group is modified with a 2 prime OMe
  modified_base 29: mod_base = OTHER; note = sugar group is a ribose
  modified_base 31: mod_base = OTHER; note = sugar group is modified with a 2 prime OMe
  modified_base 33: mod_base = OTHER; note = sugar group is modified with a 2 prime OMe
  modified_base 35: mod_base = OTHER; note = sugar group is a ribose
  modified_base 37: mod_base = OTHER; note = sugar group is modified with a 2 prime OMe
  modified_base 39: mod_base = OTHER; note = sugar group is modified with a 2 prime OMe

SEQ ID NO: 5 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = OTHER; mod_base = m5c; note = residue is locked nucleic acid
  modified_base 4: mod_base = m5c; mod_base = OTHER; note = residue is locked nucleic acid
  modified_base 6: mod_base = OTHER; mod_base = m5c; note = residue is locked nucleic acid
  modified_base 8: mod_base = m5c; mod_base = OTHER; note = residue is locked nucleic acid
  modified_base 10: mod_base = m5c; mod_base = OTHER; note = residue is locked nucleic acid
  modified_base 12: mod_base = m5c; mod_base = OTHER; note = residue is locked nucleic acid
  modified_base 14: mod_base = m5c; mod_base = OTHER; note = residue is locked nucleic acid
  modified_base 16: mod_base = m5c
  modified_base 18: mod_base = OTHER; mod_base = m5c; note = residue is locked nucleic acid
  modified_base 20: mod_base = m5c
  modified_base 22: mod_base = m5c
  modified_base 24: mod_base = OTHER; mod_base = m5c; note = residue is locked nucleic acid
  modified_base 26: mod_base = OTHER; mod_base = m5c; note = residue is locked nucleic acid
  modified_base 28: mod_base = m5c; mod_base = OTHER; note = residue is locked nucleic acid
  modified_base 30: mod_base = OTHER; mod_base = m5c; note = residue is locked nucleic acid
  modified_base 32: mod_base = OTHER; mod_base = m5c; note = residue is locked nucleic acid
  modified_base 34: mod_base = OTHER; mod_base = m5c; note = residue is locked nucleic acid
  modified_base 36: mod_base = m5c; mod_base = OTHER; note = residue is locked nucleic acid
  modified_base 38: mod_base = m5c; mod_base = OTHER; note = residue is locked nucleic acid
  modified_base 40: mod_base = OTHER; mod_base = m5c; note = residue is locked nucleic acid
  modified_base 3: mod_base = OTHER; note = sugar group is modified with a 2 prime OMe
  modified_base 5: mod_base = OTHER; note = sugar group is modified with a 2 prime OMe
  modified_base 7: mod_base = OTHER; note = sugar group is modified with a 2 prime OMe
  modified_base 9: mod_base = OTHER; note = sugar group is modified with a 2 prime OMe
  modified_base 13: mod_base = OTHER; note = sugar group is modified with a 2 prime OMe
  modified_base 15: mod_base = OTHER; note = sugar group is modified with a 2 prime OMe
  modified_base 25: mod_base = OTHER; note = sugar group is modified with a 2 prime OMe
  modified_base 27: mod_base = OTHER; note = sugar group is modified with a 2 prime OMe
  modified_base 29: mod_base = OTHER; note = sugar group is a ribose
  modified_base 31: mod_base = OTHER; note = sugar group is modified with a 2 prime OMe
  modified_base 33: mod_base = OTHER; note = sugar group is modified with a 2 prime OMe
  modified_base 35: mod_base = OTHER; note = sugar group is a ribose
  modified_base 37: mod_base = OTHER; note = sugar group is modified with a 2 prime OMe
  modified_base 39: mod_base = OTHER; note = sugar group is modified with a 2 prime OMe

SEQ ID NO: 6 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = m5c; mod_base = OTHER; note = residue is locked nucleic acid
  modified_base 4: mod_base = m5c; mod_base = OTHER; note = residue is locked nucleic acid
  modified_base 6: mod_base = m5c; mod_base = OTHER; note = residue is locked nucleic acid
  modified_base 8: mod_base = OTHER; mod_base = m5c; note = residue is locked nucleic acid
  modified_base 10: mod_base = m5c; mod_base = OTHER; note = residue is locked nucleic acid
  modified_base 12: mod_base = OTHER; mod_base = m5c; note = residue is locked nucleic acid
  modified_base 14: mod_base = OTHER; mod_base = m5c; note = residue is locked nucleic acid
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = OTHER; mod_base = m5c; note = residue is locked nucleic acid
  modified_base 28: mod_base = OTHER; mod_base = m5c; note = residue is locked nucleic acid
  modified_base 30: mod_base = m5c; mod_base = OTHER; note = residue is locked nucleic acid
  modified_base 32: mod_base = m5c; mod_base = OTHER; note = residue is locked nucleic acid
  modified_base 34: mod_base = OTHER; mod_base = m5c; note = residue is locked nucleic acid
  modified_base 36: mod_base = m5c; mod_base = OTHER; note = residue is locked nucleic acid
  modified_base 38: mod_base = OTHER; mod_base = m5c; note = residue is locked nucleic acid
  modified_base 40: mod_base = OTHER; mod_base = m5c; note = residue is locked nucleic acid
  modified_base 3: mod_base = OTHER; note = sugar group is modified with a 2 prime OMe
  modified_base 5: mod_base = OTHER; note = sugar group is modified with a 2 prime OMe
  modified_base 7: mod_base = OTHER; note = sugar group is modified with a 2 prime OMe
  modified_base 9: mod_base = OTHER; note = sugar group is modified with a 2 prime OMe
  modified_base 13: mod_base = OTHER; note = sugar group is modified with a 2 prime OMe
  modified_base 15..16: mod_base = OTHER; note = sugar group is modified with a 2 prime OMe
  modified_base 24..25: mod_base = OTHER; note = sugar group is modified with a 2 prime OMe
  modified_base 27: mod_base = OTHER; note = sugar group is modified with a 2 prime OMe
  modified_base 29: mod_base = OTHER; note = sugar group is a ribose
  modified_base 31: mod_base = OTHER; note = sugar group is modified with a 2 prime OMe
  modified_base 33: mod_base = OTHER; note = sugar group is modified with a 2 prime OMe
  modified_base 35: mod_base = OTHER; note = sugar group is a ribose
  modified_base 37: mod_base = OTHER; note = sugar group is modified with a 2 prime OMe
  modified_base 39: mod_base = OTHER; note = sugar group is modified with a 2 prime OMe

SEQ ID NO: 7 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = OTHER; mod_base = m5c; note = residue is locked nucleic acid
  modified_base 4: mod_base = OTHER; mod_base = m5c; note = residue is locked nucleic acid
  modified_base 6: mod_base = m5c; mod_base = OTHER; note = residue is locked nucleic acid
  modified_base 8: mod_base = m5c; mod_base = OTHER; note = residue is locked nucleic acid
  modified_base 10: mod_base = OTHER; mod_base = m5c; note = residue is locked nucleic acid
  modified_base 12: mod_base = m5c; mod_base = OTHER; note = residue is locked nucleic acid
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c; mod_base = OTHER; note = residue is locked nucleic acid
  modified_base 30: mod_base = OTHER; mod_base = m5c; note = residue is locked nucleic acid
  modified_base 32: mod_base = m5c; mod_base = OTHER; note = residue is locked nucleic acid
  modified_base 34: mod_base = m5c; mod_base = OTHER; note = residue is locked nucleic acid
  modified_base 36: mod_base = OTHER; mod_base = m5c; note = residue is locked nucleic acid
  modified_base 38: mod_base = OTHER; mod_base = m5c; note = residue is locked nucleic acid
  modified_base 40: mod_base = m5c; mod_base = OTHER; note = residue is locked nucleic acid
  modified_base 3: mod_base = OTHER; note = sugar group is modified with a 2 prime OMe
  modified_base 5: mod_base = OTHER; note = sugar group is modified with a 2 prime OMe
  modified_base 7: mod_base = OTHER; note = sugar group is modified with a 2 prime OMe
  modified_base 9: mod_base = OTHER; note = sugar group is modified with a 2 prime OMe
  modified_base 13..16: mod_base = OTHER; note = sugar group is modified with a 2 prime OMe
  modified_base 24..27: mod_base = OTHER; note = sugar group is modified with a 2 prime OMe
  modified_base 29: mod_base = OTHER; note = sugar group is a ribose
  modified_base 31: mod_base = OTHER; note = sugar group is modified with a 2 prime OMe
  modified_base 33: mod_base = OTHER; note = sugar group is modified with a 2 prime OMe
  modified_base 35: mod_base = OTHER; note = sugar group is a ribose
  modified_base 37: mod_base = OTHER; note = sugar group is modified with a 2 prime OMe
  modified_base 39: mod_base = OTHER; note = sugar group is modified with a 2 prime OMe

SEQ ID NO: 8 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = m5c; mod_base = OTHER; note = residue is locked nucleic acid
  modified_base 4: mod_base = m5c; mod_base = OTHER; note = residue is locked nucleic acid
  modified_base 6: mod_base = m5c; mod_base = OTHER; note = residue is locked nucleic acid
  modified_base 8: mod_base = OTHER; mod_base = m5c; note = residue is locked nucleic acid
  modified_base 10: mod_base = OTHER; mod_base = m5c; note = residue is locked nucleic acid
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = OTHER; mod_base = m5c; note = residue is locked nucleic acid
  modified_base 32: mod_base = OTHER; mod_base = m5c; note = residue is locked nucleic acid
  modified_base 34: mod_base = m5c; mod_base = OTHER; note = residue is locked nucleic acid
  modified_base 36: mod_base = OTHER; mod_base = m5c; note = residue is locked nucleic acid
  modified_base 38: mod_base = OTHER; mod_base = m5c; note = residue is locked nucleic acid
  modified_base 40: mod_base = OTHER; mod_base = m5c; note = residue is locked nucleic acid
  modified_base 3: mod_base = OTHER; note = sugar group is modified with a 2 prime OMe
  modified_base 5: mod_base = OTHER; note = sugar group is modified with a 2 prime OMe
  modified_base 7: mod_base = OTHER; note = sugar group is modified with a 2 prime OMe
  modified_base 9: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 31: mod_base = OTHER; note = sugar group is modified with a 2 prime OMe
  modified_base 33: mod_base = OTHER; note = sugar group is modified with a 2 prime OMe
  modified_base 35: mod_base = OTHER; note = sugar group is a ribose
  modified_base 37: mod_base = OTHER; note = sugar group is modified with a 2 prime OMe
  modified_base 39: mod_base = OTHER; note = sugar group is modified with a 2 prime OMe

SEQ ID NO: 9 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = m5c; mod_base = OTHER; note = residue is locked nucleic acid
  modified_base 4: mod_base = m5c; mod_base = OTHER; note = residue is locked nucleic acid
  modified_base 6: mod_base = m5c; mod_base = OTHER; note = residue is locked nucleic acid
  modified_base 8: mod_base = OTHER; mod_base = m5c; note = residue is locked nucleic acid
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c; mod_base = OTHER; note = residue is locked nucleic acid
  modified_base 34: mod_base = m5c; mod_base = OTHER; note = residue is locked nucleic acid
  modified_base 36: mod_base = m5c; mod_base = OTHER; note = residue is locked nucleic acid
  modified_base 38: mod_base = OTHER; mod_base = m5c; note = residue is locked nucleic acid
  modified_base 40: mod_base = OTHER; mod_base = m5c; note = residue is locked nucleic acid
  modified_base 3: mod_base = OTHER; note = sugar group is modified with a 2 prime OMe
  modified_base 5: mod_base = OTHER; note = sugar group is modified with a 2 prime OMe
  modified_base 7: mod_base = OTHER; note = sugar group is modified with a 2 prime OMe
  modified_base 9..10: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 30..31: mod_base = OTHER; note = sugar group is modified with a 2 prime OMe
  modified_base 33: mod_base = OTHER; note = sugar group is modified with a 2 prime OMe
  modified_base 35: mod_base = OTHER; note = sugar group is a ribose
  modified_base 37: mod_base = OTHER; note = sugar group is modified with a 2 prime OMe
  modified_base 39: mod_base = OTHER; note = sugar group is modified with a 2 prime OMe

SEQ ID NO: 10 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = m5c; mod_base = OTHER; note = residue is locked nucleic acid
  modified_base 4: mod_base = m5c; mod_base = OTHER; note = residue is locked nucleic acid
  modified_base 6: mod_base = m5c; mod_base = OTHER; note = residue is locked nucleic acid
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c; mod_base = OTHER; note = residue is locked nucleic acid
  modified_base 36: mod_base = OTHER; mod_base = m5c; note = residue is locked nucleic acid
  modified_base 38: mod_base = m5c; mod_base = OTHER; note = residue is locked nucleic acid
  modified_base 40: mod_base = OTHER; mod_base = m5c; note = residue is locked nucleic acid
  modified_base 3: mod_base = OTHER; note = sugar group is modified with a 2 prime OMe
  modified_base 5: mod_base = OTHER; note = sugar group is modified with a 2 prime OMe
  modified_base 7..10: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 30..33: mod_base = OTHER; note = sugar group is modified with a 2 prime OMe
  modified_base 35: mod_base = OTHER; note = sugar group is a ribose
  modified_base 37: mod_base = OTHER; note = sugar group is modified with a 2 prime OMe
  modified_base 39: mod_base = OTHER; note = sugar group is modified with a 2 prime OMe

SEQ ID NO: 11 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = m5c; mod_base = OTHER; note = residue is locked nucleic acid
  modified_base 4: mod_base = OTHER; mod_base = m5c; note = residue is locked nucleic acid
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c; mod_base = OTHER; note = residue is locked nucleic acid
  modified_base 38: mod_base = OTHER; mod_base = m5c; note = residue is locked nucleic acid
  modified_base 40: mod_base = OTHER; mod_base = m5c; note = residue is locked nucleic acid
  modified_base 3: mod_base = OTHER; note = sugar group is modified with a 2 prime OMe
  modified_base 5..10: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 30..34: mod_base = OTHER; note = sugar group is modified with a 2 prime OMe
  modified_base 35: mod_base = OTHER; note = sugar group is a ribose
  modified_base 37: mod_base = OTHER; note = sugar group is modified with a 2 prime OMe
  modified_base 39: mod_base = OTHER; note = sugar group is modified with a 2 prime OMe

SEQ ID NO: 12 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = OTHER; mod_base = m5c; note = residue is locked nucleic acid
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = m5c; mod_base = OTHER; note = residue is locked nucleic acid
  modified_base 40: mod_base = m5c; mod_base = OTHER; note = residue is locked nucleic acid
  modified_base 3..10: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 30..34: mod_base = OTHER; note = sugar group is modified with a 2 prime OMe
  modified_base 35: mod_base = OTHER; note = sugar group is a ribose
  modified_base 36..37: mod_base = OTHER; note = sugar group is modified with a 2 prime OMe
  modified_base 39: mod_base = OTHER; note = sugar group is modified with a 2 prime OMe

SEQ ID NO: 13 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = m5c
  modified_base 40: mod_base = OTHER; mod_base = m5c; note = residue is locked nucleic acid
  modified_base 1..10: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 30..34: mod_base = OTHER; note = sugar group is modified with a 2 prime OMe
  modified_base 35: mod_base = OTHER; note = sugar group is a ribose
  modified_base 36..39: mod_base = OTHER; note = sugar group is modified with a 2 prime OMe

SEQ ID NO: 14 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = m5c; mod_base = OTHER; note = residue is locked nucleic acid
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = m5c
  modified_base 40: mod_base = m5c
  modified_base 3..10: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 30..34: mod_base = OTHER; note = sugar group is modified with a 2 prime OMe
  modified_base 35: mod_base = OTHER; note = sugar group is a ribose
  modified_base 36..40: mod_base = OTHER; note = sugar group is modified with a 2 prime OMe

SEQ ID NO: 15 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = OTHER; mod_base = m5c; note = residue is locked nucleic acid
  modified_base 4: mod_base = m5c; mod_base = OTHER; note = residue is locked nucleic acid
  modified_base 6: mod_base = OTHER; mod_base = m5c; note = residue is locked nucleic acid
  modified_base 8: mod_base = OTHER; mod_base = m5c; note = residue is locked nucleic acid
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = OTHER; mod_base = m5c; note = residue is locked nucleic acid
  modified_base 36: mod_base = m5c; mod_base = OTHER; note = residue is locked nucleic acid
  modified_base 38: mod_base = OTHER; mod_base = m5c; note = residue is locked nucleic acid
  modified_base 40: mod_base = m5c; mod_base = OTHER; note = residue is locked nucleic acid
  modified_base 3: mod_base = OTHER; note = sugar group is modified with a 2 prime OMe
  modified_base 5: mod_base = OTHER; note = sugar group is modified with a 2 prime OMe
  modified_base 7: mod_base = OTHER; note = sugar group is modified with a 2 prime OMe
  modified_base 9..10: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 30..33: mod_base = OTHER; note = sugar group is modified with a 2 prime OMe
  modified_base 35: mod_base = OTHER; note = sugar group is a ribose
  modified_base 37: mod_base = OTHER; note = sugar group is modified with a 2 prime OMe
  modified_base 39: mod_base = OTHER; note = sugar group is modified with a 2 prime OMe

SEQ ID NO: 16 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = OTHER; mod_base = m5c; note = residue is locked nucleic acid
  modified_base 4: mod_base = OTHER; mod_base = m5c; note = residue is locked nucleic acid
  modified_base 6: mod_base = m5c; mod_base = OTHER; note = residue is locked nucleic acid
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = OTHER; mod_base = m5c; note = residue is locked nucleic acid
  modified_base 38: mod_base = m5c; mod_base = OTHER; note = residue is locked nucleic acid
  modified_base 40: mod_base = OTHER; mod_base = m5c; note = residue is locked nucleic acid
  modified_base 3: mod_base = OTHER; note = sugar group is modified with a 2 prime OMe
  modified_base 5: mod_base = OTHER; note = sugar group is modified with a 2 prime OMe
  modified_base 7..10: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 30..34: mod_base = OTHER; note = sugar group is modified with a 2 prime OMe
  modified_base 35: mod_base = OTHER; note = sugar group is a ribose
  modified_base 37: mod_base = OTHER; note = sugar group is modified with a 2 prime OMe
  modified_base 39: mod_base = OTHER; note = sugar group is modified with a 2 prime OMe

SEQ ID NO: 17 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = OTHER; mod_base = m5c; note = residue is locked nucleic acid
  modified_base 4: mod_base = OTHER; mod_base = m5c; note = residue is locked nucleic acid
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = m5c; mod_base = OTHER; note = residue is locked nucleic acid
  modified_base 40: mod_base = OTHER; mod_base = m5c; note = residue is locked nucleic acid
  modified_base 3: mod_base = OTHER; note = sugar group is modified with a 2 prime OMe
  modified_base 5..10: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 30..34: mod_base = OTHER; note = sugar group is modified with a 2 prime OMe
  modified_base 35: mod_base = OTHER; note = sugar group is a ribose
  modified_base 36..37: mod_base = OTHER; note = sugar group is modified with a 2 prime OMe
  modified_base 39: mod_base = OTHER; note = sugar group is modified with a 2 prime OMe

SEQ ID NO: 18 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = OTHER; mod_base = m5c; note = residue is locked nucleic acid
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = m5c
  modified_base 40: mod_base = m5c; mod_base = OTHER; note = residue is locked nucleic acid
  modified_base 3..10: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 30..34: mod_base = OTHER; note = sugar group is modified with a 2 prime OMe
  modified_base 35: mod_base = OTHER; note = sugar group is a ribose
  modified_base 36..39: mod_base = OTHER; note = sugar group is modified with a 2 prime OMe

SEQ ID NO: 19 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = m5c; mod_base = OTHER; note = residue is locked nucleic acid
  modified_base 4: mod_base = m5c; mod_base = OTHER; note = residue is locked nucleic acid
  modified_base 6: mod_base = m5c; mod_base = OTHER; note = residue is locked nucleic acid
  modified_base 8: mod_base = m5c; mod_base = OTHER; note = residue is locked nucleic acid
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = OTHER; mod_base = m5c; note = residue is locked nucleic acid
  modified_base 38: mod_base = OTHER; mod_base = m5c; note = residue is locked nucleic acid
  modified_base 40: mod_base = m5c; mod_base = OTHER; note = residue is locked nucleic acid
  modified_base 3: mod_base = OTHER; note = sugar group is modified with a 2 prime OMe
  modified_base 5: mod_base = OTHER; note = sugar group is modified with a 2 prime OMe
  modified_base 7: mod_base = OTHER; note = sugar group is modified with a 2 prime OMe
  modified_base 9..10: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 30..34: mod_base = OTHER; note = sugar group is modified with a 2 prime OMe
  modified_base 35: mod_base = OTHER; note = sugar group is a ribose
  modified_base 37: mod_base = OTHER; note = sugar group is modified with a 2 prime OMe
  modified_base 39: mod_base = OTHER; note = sugar group is modified with a 2 prime OMe

SEQ ID NO: 20 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = m5c; mod_base = OTHER; note = residue is locked nucleic acid
  modified_base 4: mod_base = OTHER; mod_base = m5c; note = residue is locked nucleic acid
  modified_base 6: mod_base = m5c; mod_base = OTHER; note = residue is locked nucleic acid
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = OTHER; mod_base = m5c; note = residue is locked nucleic acid
  modified_base 40: mod_base = m5c; mod_base = OTHER; note = residue is locked nucleic acid
  modified_base 3: mod_base = OTHER; note = sugar group is modified with a 2 prime OMe
  modified_base 5: mod_base = OTHER; note = sugar group is modified with a 2 prime OMe
  modified_base 7..10: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 30..34: mod_base = OTHER; note = sugar group is modified with a 2 prime OMe
  modified_base 35: mod_base = OTHER; note = sugar group is a ribose
  modified_base 36..37: mod_base = OTHER; note = sugar group is modified with a 2 prime OMe
  modified_base 39: mod_base = OTHER; note = sugar group is modified with a 2 prime OMe

SEQ ID NO: 21 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = OTHER; mod_base = m5c; note = residue is locked nucleic acid
  modified_base 4: mod_base = OTHER; mod_base = m5c; note = residue is locked nucleic acid
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = m5c
  modified_base 40: mod_base = m5c; mod_base = OTHER; note = residue is locked nucleic acid
  modified_base 3: mod_base = OTHER; note = sugar group is modified with a 2 prime OMe
  modified_base 5..10: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 30..34: mod_base = OTHER; note = sugar group is modified with a 2 prime OMe
  modified_base 35: mod_base = OTHER; note = sugar group is a ribose
  modified_base 36..39: mod_base = OTHER; note = sugar group is modified with a 2 prime OMe
  modified_base 39: mod_base = OTHER; note = sugar group is modified with a 2 prime OMe

SEQ ID NO: 22 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = m5c; mod_base = OTHER; note = residue is locked nucleic acid
  modified_base 4: mod_base = m5c; mod_base = OTHER; note = residue is locked nucleic acid
  modified_base 6: mod_base = m5c; mod_base = OTHER; note = residue is locked nucleic acid
  modified_base 8: mod_base = m5c; mod_base = OTHER; note = residue is locked nucleic acid
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = m5c
  modified_base 40: mod_base = m5c
  modified_base 3: mod_base = OTHER; note = sugar group is modified with a 2 prime OMe
  modified_base 5: mod_base = OTHER; note = sugar group is modified with a 2 prime OMe
  modified_base 7: mod_base = OTHER; note = sugar group is modified with a 2 prime OMe
  modified_base 9..10: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 30..34: mod_base = OTHER; note = sugar group is modified with a 2 prime OMe
  modified_base 35: mod_base = OTHER; note = sugar group is a ribose
  modified_base 36..40: mod_base = OTHER; note = sugar group is modified with a 2 prime OMe

SEQ ID NO: 23 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1..10: mod_base = OTHER; note = sugar group is modified with a 2 prime OMe
  modified_base 11: mod_base = OTHER; note = sugar group is a ribose
  modified_base 12..16: mod_base = OTHER; note = sugar group is modified with a 2 prime OMe
  modified_base 17: mod_base = OTHER; note = sugar group is a ribose
  modified_base 24..28: mod_base = OTHER; note = sugar group is modified with a 2 prime OMe
  modified_base 23: mod_base = OTHER; note = sugar group is a ribose
  modified_base 30..33: mod_base = OTHER; note = sugar group is modified with a 2 prime OMe
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = OTHER; mod_base = m5c; note = residue is locked nucleic acid
  modified_base 36: mod_base = m5c; mod_base = OTHER; note = residue is locked nucleic acid
  modified_base 38: mod_base = OTHER; mod_base = m5c; note = residue is locked nucleic acid
  modified_base 40: mod_base = m5c; mod_base = OTHER; note = residue is locked nucleic acid
  modified_base 18..22: mod_base = OTHER; note = sugar group is modified with a 2 prime OMe
  modified_base 29: mod_base = OTHER; note = sugar group is a ribose
  modified_base 35: mod_base = OTHER; note = sugar group is a ribose
  modified_base 37: mod_base = OTHER; note = sugar group is modified with a 2 prime OMe
  modified_base 39: mod_base = OTHER; note = sugar group is modified with a 2 prime OMe

SEQ ID NO: 24 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = m5c; mod_base = OTHER; note = residue is locked nucleic acid
  modified_base 4: mod_base = m5c; mod_base = OTHER; note = residue is locked nucleic acid
  modified_base 6: mod_base = m5c; mod_base = OTHER; note = residue is locked nucleic acid
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = m5c
  modified_base 40: mod_base = m5c
  modified_base 3: mod_base = OTHER; note = sugar group is modified with a 2 prime OMe
  modified_base 5: mod_base = OTHER; note = sugar group is modified with a 2 prime OMe
  modified_base 7..10: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 30..34: mod_base = OTHER; note = sugar group is modified with a 2 prime OMe
  modified_base 35: mod_base = OTHER; note = sugar group is a ribose
  modified_base 36..40: mod_base = OTHER; note = sugar group is modified with a 2 prime OMe
  modified_base 39: mod_base = OTHER; note = sugar group is modified with a 2 prime OMe

SEQ ID NO: 25 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1..10: mod_base = OTHER; note = sugar group is modified with a 2 prime OMe
  modified_base 11: mod_base = OTHER; note = sugar group is a ribose
  modified_base 12..16: mod_base = OTHER; note = sugar group is modified with a 2 prime OMe
  modified_base 17: mod_base = OTHER; note = sugar group is a ribose
  modified_base 24..28: mod_base = OTHER; note = sugar group is modified with a 2 prime OMe
  modified_base 23: mod_base = OTHER; note = sugar group is a ribose
  modified_base 30..34: mod_base = OTHER; note = sugar group is modified with a 2 prime OMe
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = OTHER; mod_base = m5c; note = residue is locked nucleic acid
  modified_base 38: mod_base = OTHER; mod_base = m5c; note = residue is locked nucleic acid
  modified_base 40: mod_base = OTHER; mod_base = m5c; note = residue is locked nucleic acid
  modified_base 18..22: mod_base = OTHER; note = sugar group is modified with a 2 prime OMe
  modified_base 29: mod_base = OTHER; note = sugar group is a ribose
  modified_base 35: mod_base = OTHER; note = sugar group is a ribose
  modified_base 37: mod_base = OTHER; note = sugar group is modified with a 2 prime OMe
  modified_base 39: mod_base = OTHER; note = sugar group is modified with a 2 prime OMe

SEQ ID NO: 26 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = OTHER; mod_base = m5c; note = residue is locked nucleic acid
  modified_base 4: mod_base = m5c; mod_base = OTHER; note = residue is locked nucleic acid
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = m5c; mod_base = OTHER; note = sugar group is modified with a 2 prime OMe
  modified_base 40: mod_base = m5c
  modified_base 3: mod_base = OTHER; note = sugar group is modified with a 2 prime OMe
  modified_base 5..10: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 30..34: mod_base = OTHER; note = sugar group is modified with a 2 prime OMe
  modified_base 35: mod_base = OTHER; note = sugar group is a ribose
  modified_base 36..40: mod_base = OTHER; note = sugar group is modified with a 2 prime OMe
  modified_base 39: mod_base = OTHER; note = sugar group is modified with a 2 prime OMe

SEQ ID NO: 27 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1..10: mod_base = OTHER; note = sugar group is modified with a 2 prime OMe
  modified_base 11: mod_base = OTHER; note = sugar group is a ribose
  modified_base 12..16: mod_base = OTHER; note = sugar group is modified with a 2 prime OMe
  modified_base 17: mod_base = OTHER; note = sugar group is a ribose
  modified_base 24..28: mod_base = OTHER; note = sugar group is modified with a 2 prime OMe
  modified_base 23: mod_base = OTHER; note = sugar group is a ribose
  modified_base 30..34: mod_base = OTHER; note = sugar group is modified with a 2 prime OMe
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = OTHER; mod_base = m5c; note = residue is locked nucleic acid
  modified_base 40: mod_base = m5c; mod_base = OTHER; note = residue is locked nucleic acid
  modified_base 18..22: mod_base = OTHER; note = sugar group is modified with a 2 prime OMe
  modified_base 29: mod_base = OTHER; note = sugar group is a ribose
  modified_base 35: mod_base = OTHER; note = sugar group is a ribose
  modified_base 36..37: mod_base = OTHER; note = sugar group is modified with a 2 prime OMe
  modified_base 39: mod_base = OTHER; note = sugar group is modified with a 2 prime OMe

SEQ ID NO: 28 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 18..22: mod_base = OTHER; note = sugar group is modified with a 2 prime OMe
  modified_base 23: mod_base = OTHER; note = sugar group is a ribose
  modified_base 2: mod_base = m5c; mod_base = OTHER; note = residue is locked nucleic acid
  modified_base 4: mod_base = m5c
  modified_base 6: mod_base = OTHER; mod_base = m5c; note = residue is locked nucleic acid
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = OTHER; mod_base = m5c; note = residue is locked nucleic acid
  modified_base 38: mod_base = m5c
  modified_base 40: mod_base = OTHER; mod_base = m5c; note = residue is locked nucleic acid
  modified_base 3..5: mod_base = OTHER; note = sugar group is modified with a 2 prime OMe
  modified_base 7..10: mod_base = OTHER; note = sugar group is modified with a 2 prime OMe
  modified_base 12..16: mod_base = OTHER; note = sugar group is modified with a 2 prime OMe
  modified_base 24..28: mod_base = OTHER; note = sugar group is modified with a 2 prime OMe
  modified_base 29: mod_base = OTHER; note = sugar group is a ribose
  modified_base 30..34: mod_base = OTHER; note = sugar group is modified with a 2 prime OMe
  modified_base 35: mod_base = OTHER; note = sugar group is a ribose
  modified_base 37..39: mod_base = OTHER; note = sugar group is modified with a 2 prime OMe
  modified_base 39: mod_base = OTHER; note = sugar group is modified with a 2 prime OMe

SEQ ID NO: 29 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1..3: mod_base = OTHER; note = sugar group is modified with a 2 prime OMe
  modified_base 11: mod_base = OTHER; note = sugar group is a ribose
  modified_base 12..16: mod_base = OTHER; note = sugar group is modified with a 2 prime OMe
  modified_base 17: mod_base = OTHER; note = sugar group is a ribose
  modified_base 24..28: mod_base = OTHER; note = sugar group is modified with a 2 prime OMe
  modified_base 23: mod_base = OTHER; note = sugar group is a ribose
  modified_base 30..34: mod_base = OTHER; note = sugar group is modified with a 2 prime OMe
  modified_base 2: mod_base = m5c
  modified_base 4: mod_base = OTHER; mod_base = m5c; note = residue is locked nucleic acid
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = OTHER; mod_base = m5c; note = residue is locked nucleic acid
  modified_base 40: mod_base = m5c
  modified_base 18..22: mod_base = OTHER; note = sugar group is modified with a 2 prime OMe
  modified_base 29: mod_base = OTHER; note = sugar group is a ribose
  modified_base 35: mod_base = OTHER; note = sugar group is a ribose
  modified_base 36..37: mod_base = OTHER; note = sugar group is modified with a 2 prime OMe
  modified_base 39..40: mod_base = OTHER; note = sugar group is modified with a 2 prime OMe
  modified_base 5..10: mod_base = OTHER; note = sugar group is modified with a 2 prime OMe

SEQ ID NO: 30 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 12..16: mod_base = OTHER; note = sugar group is modified with a 2 prime OMe
  modified_base 17: mod_base = OTHER; note = sugar group is a ribose
  modified_base 24..28: mod_base = OTHER; note = sugar group is modified with a 2 prime OMe
  modified_base 23: mod_base = OTHER; note = sugar group is a ribose
  modified_base 30..34: mod_base = OTHER; note = sugar group is modified with a 2 prime OMe
  modified_base 2: mod_base = OTHER; mod_base = m5c; note = residue is locked nucleic acid
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = OTHER; mod_base = m5c; note = residue is locked nucleic acid
  modified_base 40: mod_base = m5c
  modified_base 18..22: mod_base = OTHER; note = sugar group is modified with a 2 prime OMe
  modified_base 29: mod_base = OTHER; note = sugar group is a ribose
  modified_base 35: mod_base = OTHER; note = sugar group is a ribose
  modified_base 36..37: mod_base = OTHER; note = sugar group is modified with a 2 prime OMe
  modified_base 39..40: mod_base = OTHER; note = sugar group is modified with a 2 prime OMe
  modified_base 3..10: mod_base = OTHER; note = sugar group is modified with a 2 prime OMe

SEQ ID NO: 31 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1..10: mod_base = OTHER; note = sugar group is modified with a 2 prime OMe
  modified_base 11: mod_base = OTHER; note = sugar group is a ribose
  modified_base 12..16: mod_base = OTHER; note = sugar group is modified with a 2 prime OMe
  modified_base 17: mod_base = OTHER; note = sugar group is a ribose
  modified_base 24..28: mod_base = OTHER; note = sugar group is modified with a 2 prime OMe
  modified_base 23: mod_base = OTHER; note = sugar group is a ribose
  modified_base 30..33: mod_base = OTHER; note = sugar group is modified with a 2 prime OMe
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c; mod_base = OTHER; note = residue is locked nucleic acid
  modified_base 36: mod_base = m5c
  modified_base 38: mod_base = OTHER; mod_base = m5c; note = residue is locked nucleic acid
  modified_base 40: mod_base = OTHER; mod_base = m5c; note = residue is locked nucleic acid
  modified_base 18..22: mod_base = OTHER; note = sugar group is modified with a 2 prime OMe
  modified_base 29: mod_base = OTHER; note = sugar group is a ribose
  modified_base 35: mod_base = OTHER; note = sugar group is a ribose
  modified_base 36..37: mod_base = OTHER; note = sugar group is modified with a 2 prime OMe
  modified_base 39: mod_base = OTHER; note = sugar group is modified with a 2 prime OMe

SEQ ID NO: 32 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1..10: mod_base = OTHER; note = sugar group is modified with a 2 prime OMe
  modified_base 11: mod_base = OTHER; note = sugar group is a ribose
  modified_base 12..16: mod_base = OTHER; note = sugar group is modified with a 2 prime OMe
  modified_base 17: mod_base = OTHER; note = sugar group is a ribose
  modified_base 24..28: mod_base = OTHER; note = sugar group is modified with a 2 prime OMe
  modified_base 23: mod_base = OTHER; note = sugar group is a ribose
  modified_base 30..33: mod_base = OTHER; note = sugar group is modified with a 2 prime OMe
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c; mod_base = OTHER; note = residue is locked nucleic acid
  modified_base 36: mod_base = OTHER; mod_base = m5c; note = residue is locked nucleic acid
  modified_base 38: mod_base = m5c
  modified_base 40: mod_base = OTHER; mod_base = m5c; note = residue is locked nucleic acid
  modified_base 18..22: mod_base = OTHER; note = sugar group is modified with a 2 prime OMe
  modified_base 29: mod_base = OTHER; note = sugar group is a ribose
  modified_base 35: mod_base = OTHER; note = sugar group is a ribose
  modified_base 37..39: mod_base = OTHER; note = sugar group is modified with a 2 prime OMe

SEQ ID NO: 33 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1..10: mod_base = OTHER; note = sugar group is modified with a 2 prime OMe
  modified_base 11: mod_base = OTHER; note = sugar group is a ribose
  modified_base 12..16: mod_base = OTHER; note = sugar group is modified with a 2 prime OMe
  modified_base 17: mod_base = OTHER; note = sugar group is a ribose
  modified_base 24..28: mod_base = OTHER; note = sugar group is modified with a 2 prime OMe
  modified_base 23: mod_base = OTHER; note = sugar group is a ribose
  modified_base 30..34: mod_base = OTHER; note = sugar group is modified with a 2 prime OMe
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = OTHER; mod_base = m5c; note = residue is locked nucleic acid
  modified_base 38: mod_base = m5c
  modified_base 40: mod_base = m5c; mod_base = OTHER; note = residue is locked nucleic acid
  modified_base 18..22: mod_base = OTHER; note = sugar group is modified with a 2 prime OMe
  modified_base 29: mod_base = OTHER; note = sugar group is a ribose
  modified_base 35: mod_base = OTHER; note = sugar group is a ribose
  modified_base 37..39: mod_base = OTHER; note = sugar group is modified with a 2 prime OMe

SEQ ID NO: 34 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1..10: mod_base = OTHER; note = sugar group is modified with a 2 prime OMe
  modified_base 11: mod_base = OTHER; note = sugar group is a ribose
  modified_base 12..16: mod_base = OTHER; note = sugar group is modified with a 2 prime OMe
  modified_base 17: mod_base = OTHER; note = sugar group is a ribose
  modified_base 24..28: mod_base = OTHER; note = sugar group is modified with a 2 prime OMe
  modified_base 23: mod_base = OTHER; note = sugar group is a ribose
  modified_base 30..33: mod_base = OTHER; note = sugar group is modified with a 2 prime OMe
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = OTHER; mod_base = m5c; note = residue is locked nucleic acid
  modified_base 36: mod_base = OTHER; mod_base = m5c; note = residue is locked nucleic acid
  modified_base 38: mod_base = m5c; mod_base = OTHER; note = residue is locked nucleic acid
  modified_base 40: mod_base = m5c
  modified_base 18..22: mod_base = OTHER; note = sugar group is modified with a 2 prime OMe
  modified_base 29: mod_base = OTHER; note = sugar group is a ribose
  modified_base 35: mod_base = OTHER; note = sugar group is a ribose
  modified_base 37: mod_base = OTHER; note = sugar group is modified with a 2 prime OMe
  modified_base 39: mod_base = OTHER; note = sugar group is modified with a 2 prime OMe

SEQ ID NO: 35 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1..10: mod_base = OTHER; note = sugar group is modified with a 2 prime OMe
  modified_base 11: mod_base = OTHER; note = sugar group is a ribose
  modified_base 12..16: mod_base = OTHER; note = sugar group is modified with a 2 prime OMe
  modified_base 17: mod_base = OTHER; note = sugar group is a ribose
  modified_base 24..28: mod_base = OTHER; note = sugar group is modified with a 2 prime OMe
  modified_base 23: mod_base = OTHER; note = sugar group is a ribose
  modified_base 30..34: mod_base = OTHER; note = sugar group is modified with a 2 prime OMe
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c; mod_base = OTHER; note = residue is locked nucleic acid
  modified_base 38: mod_base = m5c; mod_base = OTHER; note = residue is locked nucleic acid
  modified_base 40: mod_base = m5c
  modified_base 18..22: mod_base = OTHER; note = sugar group is modified with a 2 prime OMe
  modified_base 29: mod_base = OTHER; note = sugar group is a ribose
  modified_base 35: mod_base = OTHER; note = sugar group is a ribose
  modified_base 37: mod_base = OTHER; note = sugar group is modified with a 2 prime OMe
  modified_base 39: mod_base = OTHER; note = sugar group is modified with a 2 prime OMe

SEQ ID NO: 36 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 21: mod_base = OTHER; note = sugar group is a ribose
  modified_base 22..30: mod_base = OTHER; note = sugar group is modified with a 2 prime OMe
  modified_base 2: mod_base = OTHER; mod_base = m5c; note = residue is locked nucleic acid
  modified_base 4: mod_base = m5c; mod_base = OTHER; note = residue is locked nucleic acid
  modified_base 6: mod_base = m5c; mod_base = OTHER; note = residue is locked nucleic acid
  modified_base 8: mod_base = m5c; mod_base = OTHER; note = residue is locked nucleic acid
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = OTHER; mod_base = m5c; note = residue is locked nucleic acid
  modified_base 34: mod_base = m5c; mod_base = OTHER; note = residue is locked nucleic acid
  modified_base 36: mod_base = OTHER; mod_base = m5c; note = residue is locked nucleic acid
  modified_base 38: mod_base = OTHER; mod_base = m5c; note = residue is locked nucleic acid
  modified_base 40: mod_base = OTHER; mod_base = m5c; note = residue is locked nucleic acid
  modified_base 3: mod_base = OTHER; note = sugar group is modified with a 2 prime OMe
  modified_base 5: mod_base = OTHER; note = sugar group is modified with a 2 prime OMe
  modified_base 7: mod_base = OTHER; note = sugar group is modified with a 2 prime OMe
  modified_base 9..10: mod_base = OTHER; note = sugar group is modified with a 2 prime OMe
  modified_base 12..20: mod_base = OTHER; note = sugar group is modified with a 2 prime OMe
  modified_base 31: mod_base = OTHER; note = sugar group is a ribose
  modified_base 33: mod_base = OTHER; note = sugar group is modified with a 2 prime OMe
  modified_base 35: mod_base = OTHER; note = sugar group is modified with a 2 prime OMe
  modified_base 37: mod_base = OTHER; note = sugar group is modified with a 2 prime OMe
  modified_base 39: mod_base = OTHER; note = sugar group is modified with a 2 prime OMe

SEQ ID NO: 37 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 21: mod_base = OTHER; note = sugar group is a ribose
  modified_base 22..30: mod_base = OTHER; note = sugar group is modified with a 2 prime OMe
  modified_base 2: mod_base = OTHER; mod_base = m5c; note = residue is locked nucleic acid
  modified_base 4: mod_base = m5c; mod_base = OTHER; note = residue is locked nucleic acid
  modified_base 6: mod_base = OTHER; mod_base = m5c; note = residue is locked nucleic acid
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = OTHER; mod_base = m5c; note = residue is locked nucleic acid
  modified_base 38: mod_base = m5c; mod_base = OTHER; note = residue is locked nucleic acid
  modified_base 40: mod_base = m5c; mod_base = OTHER; note = residue is locked nucleic acid
  modified_base 3: mod_base = OTHER; note = sugar group is modified with a 2 prime OMe
  modified_base 5: mod_base = OTHER; note = sugar group is modified with a 2 prime OMe
  modified_base 7..10: mod_base = OTHER; note = sugar group is modified with a 2 prime OMe
  modified_base 12..20: mod_base = OTHER; note = sugar group is modified with a 2 prime OMe
  modified_base 31: mod_base = OTHER; note = sugar group is a ribose
  modified_base 32..35: mod_base = OTHER; note = sugar group is modified with a 2 prime OMe
  modified_base 37: mod_base = OTHER; note = sugar group is modified with a 2 prime OMe
  modified_base 39: mod_base = OTHER; note = sugar group is modified with a 2 prime OMe

SEQ ID NO: 38 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 21: mod_base = OTHER; note = sugar group is a ribose
  modified_base 22..30: mod_base = OTHER; note = sugar group is modified with a 2 prime OMe
  modified_base 2: mod_base = OTHER; mod_base = m5c; note = residue is locked nucleic acid
  modified_base 4: mod_base = m5c; mod_base = OTHER; note = residue is locked nucleic acid
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = m5c; mod_base = OTHER; note = residue is locked nucleic acid
  modified_base 40: mod_base = OTHER; mod_base = m5c; note = residue is locked nucleic acid
  modified_base 3: mod_base = OTHER; note = sugar group is modified with a 2 prime OMe
  modified_base 5..10: mod_base = OTHER; note = sugar group is modified with a 2 prime OMe
  modified_base 12..20: mod_base = OTHER; note = sugar group is modified with a 2 prime OMe
  modified_base 31: mod_base = OTHER; note = sugar group is a ribose
  modified_base 32..37: mod_base = OTHER; note = sugar group is modified with a 2 prime OMe
  modified_base 39: mod_base = OTHER; note = sugar group is modified with a 2 prime OMe

SEQ ID NO: 39 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 3..10: mod_base = OTHER; note = sugar group is modified with a 2 prime OMe
  modified_base 11: mod_base = OTHER; note = sugar group is a ribose
  modified_base 12..20: mod_base = OTHER; note = sugar group is modified with a 2 prime OMe
  modified_base 21: mod_base = OTHER; note = sugar group is a ribose
  modified_base 22..30: mod_base = OTHER; note = sugar group is modified with a 2 prime OMe
  modified_base 31: mod_base = OTHER; note = sugar group is a ribose
  modified_base 32..39: mod_base = OTHER; note = sugar group is modified with a 2 prime OMe
  modified_base 2: mod_base = OTHER; mod_base = m5c; note = residue is locked nucleic acid
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = m5c
  modified_base 40: mod_base = OTHER; mod_base = m5c; note = residue is locked nucleic acid
  modified_base 1: mod_base = OTHER; note = sugar group is modified with a 2 prime OMe

SEQ ID NO: 40 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1..10: mod_base = OTHER; note = sugar group is modified with a 2 prime OMe
  modified_base 11: mod_base = OTHER; note = sugar group is a ribose
  modified_base 12..20: mod_base = OTHER; note = sugar group is modified with a 2 prime OMe
  modified_base 21: mod_base = OTHER; note = sugar group is a ribose
  modified_base 22..30: mod_base = OTHER; note = sugar group is modified with a 2 prime OMe
  modified_base 31: mod_base = OTHER; note = sugar group is a ribose
  modified_base 32..33: mod_base = OTHER; note = sugar group is modified with a 2 prime OMe
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = OTHER; mod_base = m5c; note = residue is locked nucleic acid
  modified_base 36: mod_base = m5c; mod_base = OTHER; note = residue is locked nucleic acid
  modified_base 38: mod_base = OTHER; mod_base = m5c; note = residue is locked nucleic acid
  modified_base 40: mod_base = m5c; mod_base = OTHER; note = residue is locked nucleic acid
  modified_base 35: mod_base = OTHER; note = sugar group is modified with a 2 prime OMe
  modified_base 37: mod_base = OTHER; note = sugar group is modified with a 2 prime OMe
  modified_base 39: mod_base = OTHER; note = sugar group is modified with a 2 prime OMe

SEQ ID NO: 41 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1..10: mod_base = OTHER; note = sugar group is modified with a 2 prime OMe
  modified_base 11: mod_base = OTHER; note = sugar group is a ribose
  modified_base 12..20: mod_base = OTHER; note = sugar group is modified with a 2 prime OMe
  modified_base 21: mod_base = OTHER; note = sugar group is a ribose
  modified_base 22..30: mod_base = OTHER; note = sugar group is modified with a 2 prime OMe
  modified_base 31: mod_base = OTHER; note = sugar group is a ribose
  modified_base 32..35: mod_base = OTHER; note = sugar group is modified with a 2 prime OMe
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c; mod_base = OTHER; note = residue is locked nucleic acid
  modified_base 38: mod_base = m5c; mod_base = OTHER; note = residue is locked nucleic acid
  modified_base 40: mod_base = OTHER; mod_base = m5c; note = residue is locked nucleic acid
  modified_base 37: mod_base = OTHER; note = sugar group is modified with a 2 prime OMe
  modified_base 39: mod_base = OTHER; note = sugar group is modified with a 2 prime OMe

SEQ ID NO: 42 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1..10: mod_base = OTHER; note = sugar group is modified with a 2 prime OMe
  modified_base 11: mod_base = OTHER; note = sugar group is a ribose
  modified_base 12..20: mod_base = OTHER; note = sugar group is modified with a 2 prime OMe
  modified_base 21: mod_base = OTHER; note = sugar group is a ribose
  modified_base 22..30: mod_base = OTHER; note = sugar group is modified with a 2 prime OMe
  modified_base 31: mod_base = OTHER; note = sugar group is a ribose
  modified_base 32..37: mod_base = OTHER; note = sugar group is modified with a 2 prime OMe
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = m5c; mod_base = OTHER; note = residue is locked nucleic acid
  modified_base 40: mod_base = OTHER; mod_base = m5c; note = residue is locked nucleic acid
  modified_base 39: mod_base = OTHER; note = sugar group is modified with a 2 prime OMe

SEQ ID NO: 43 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 2: mod_base = m5c; mod_base = OTHER; note = residue is locked nucleic acid
  modified_base 4: mod_base = OTHER; mod_base = m5c; note = residue is locked nucleic acid
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = m5c; mod_base = OTHER; note = residue is locked nucleic acid
  modified_base 40: mod_base = m5c; mod_base = OTHER; note = residue is locked nucleic acid
  modified_base 3: mod_base = OTHER; note = sugar group is modified with a 2 prime OMe
  modified_base 5..37: mod_base = OTHER; note = sugar group is modified with a 2 prime OMe
  modified_base 39: mod_base = OTHER; note = sugar group is modified with a 2 prime OMe

SEQ ID NO: 44 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1: mod_base = OTHER; note = sugar group is modified with a 2 prime OMe
  modified_base 11: mod_base = OTHER; note = sugar group is a ribose
  modified_base 17: mod_base = OTHER; note = sugar group is a ribose
  modified_base 19..21: mod_base = OTHER; note = sugar group is modified with a 2 prime OMe
  modified_base 23: mod_base = OTHER; note = sugar group is a ribose
  modified_base 2: mod_base = OTHER; mod_base = m5c; note = residue is locked nucleic acid
  modified_base 4: mod_base = m5c; mod_base = OTHER; note = residue is locked nucleic acid
  modified_base 6: mod_base = m5c; mod_base = OTHER; note = residue is locked nucleic acid
  modified_base 8: mod_base = m5c; mod_base = OTHER; note = residue is locked nucleic acid
  modified_base 10: mod_base = OTHER; mod_base = m5c; note = residue is locked nucleic acid
  modified_base 12: mod_base = OTHER; mod_base = m5c; note = residue is locked nucleic acid
  modified_base 14: mod_base = OTHER; mod_base = m5c; note = residue is locked nucleic acid
  modified_base 16: mod_base = OTHER; mod_base = m5c; note = residue is locked nucleic acid
  modified_base 18: mod_base = OTHER; mod_base = m5c; note = residue is locked nucleic acid
  modified_base 20: mod_base = m5c; mod_base = OTHER; note = residue is locked nucleic acid
  modified_base 22: mod_base = m5c; mod_base = OTHER; note = residue is locked nucleic acid
  modified_base 24: mod_base = m5c; mod_base = OTHER; note = residue is locked nucleic acid
  modified_base 26: mod_base = OTHER; mod_base = m5c; note = residue is locked nucleic acid
  modified_base 28: mod_base = OTHER; mod_base = m5c; note = residue is locked nucleic acid
  modified_base 30: mod_base = m5c; mod_base = OTHER; note = residue is locked nucleic acid
  modified_base 32: mod_base = m5c; mod_base = OTHER; note = residue is locked nucleic acid
  modified_base 34: mod_base = m5c; mod_base = OTHER; note = residue is locked nucleic acid
  modified_base 36: mod_base = m5c; mod_base = OTHER; note = residue is locked nucleic acid
  modified_base 38: mod_base = OTHER; mod_base = m5c; note = residue is locked nucleic acid
  modified_base 40: mod_base = m5c; mod_base = OTHER; note = residue is locked nucleic acid
  modified_base 3: mod_base = OTHER; note = sugar group is modified with a 2 prime OMe
  modified_base 5: mod_base = OTHER; note = sugar group is modified with a 2 prime OMe
  modified_base 7: mod_base = OTHER; note = sugar group is modified with a 2 prime OMe
  modified_base 9: mod_base = OTHER; note = sugar group is modified with a 2 prime OMe
  modified_base 13: mod_base = OTHER; note = sugar group is modified with a 2 prime OMe
  modified_base 15: mod_base = OTHER; note = sugar group is modified with a 2 prime OMe
  modified_base 25: mod_base = OTHER; note = sugar group is modified with a 2 prime OMe
  modified_base 27: mod_base = OTHER; note = sugar group is modified with a 2 prime OMe
  modified_base 29: mod_base = OTHER; note = sugar group is a ribose
  modified_base 31: mod_base = OTHER; note = sugar group is modified with a 2 prime OMe
  modified_base 33: mod_base = OTHER; note = sugar group is modified with a 2 prime OMe
  modified_base 35: mod_base = OTHER; note = sugar group is a ribose
  modified_base 37: mod_base = OTHER; note = sugar group is modified with a 2 prime OMe
  modified_base 39: mod_base = OTHER; note = sugar group is modified with a 2 prime OMe

SEQ ID NO: 45 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1..40: mod_base = OTHER; note = residue is linked nucleic acid
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = m5c
  modified_base 40: mod_base = m5c

SEQ ID NO: 46 - DNA, 40 bp, synthetic construct; identical to SEQ ID NO: 3
  source 1..40: mol_type = other DNA
  modified_base 1..40: mod_base = OTHER; note = residues comprise a phosphorothioate internucleotide linkage
  modified_base 1..40: mod_base = OTHER; note = sugar group is modified with a 2 prime OMe
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c
  modified_base 32: mod_base = m5c
  modified_base 34: mod_base = m5c
  modified_base 36: mod_base = m5c
  modified_base 38: mod_base = m5c
  modified_base 40: mod_base = m5c

(19) United States
(12) Patent Application Publication
(10) Pub. No.: US 2025/0382618 A1
(43) Pub. Date: Dec. 18, 2025

(54) METHODS AND COMPOSITIONS FOR INHIBITING VIRUS

(71) Applicant: Aligos Therapeutics, Inc., South San Francisco, CA (US)

(72) Inventors: Santhosh Kumar Thatikonda, Fremont, CA (US); Leonid Beigelman, San Mateo, CA (US); David Bernard Smith, San Mateo, CA (US); Yannick Debing, Bilzen (BE); Lawrence M. Blatt, Healdsburg, CA (US); Jin Hong, Brisbane, CA (US); Vivek Kumar Rajwanshi, Cupertino, CA (US)

(21) Appl. No.: 19/231,307

(22) Filed: Jun. 6, 2025

Related U.S. Application Data
(60) Provisional application No. 63/660,298, filed on Jun. 14, 2024.

(51) Int. Cl. C12N 15/113; A61K 31/522; A61P 31/20

(52) U.S. Cl. CPC C12N 15/1131; A61K 31/522; A61P 31/20; C12N 2310/11; C12N 2310/14; C12N 2310/315; C12N 2310/3231

(57) ABSTRACT

The present application relates to derivatives of STOPS™ antiviral compounds having enhanced helicity, wherein the compounds are S-antigen transport inhibiting oligonucleotide polymers. This application further relates to processes for making the compounds and methods of using them to treat diseases and conditions. In some embodiments, the diseases and conditions are related to hepatitis B and/or hepatitis D.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application No. 63/660,298, filed Jun. 14, 2024, which is hereby incorporated by reference in its entirety.

### REFERENCE TO SEQUENCE LISTING

The present application is being filed along with a Sequence Listing in electronic format. The Sequence Listing is provided as a file entitled ALIG117A.xml, which was created and last modified on Jun. 5, 2025, and is 780,371 bytes in size. The information in the electronic Sequence Listing is hereby incorporated by reference in its entirety.

### BACKGROUND

### Field

This application relates to derivatives of S-antigen transport inhibiting oligonucleotide polymers (STOPS™) antiviral compounds with enhanced helicity. This application further relates to processes for making the compounds and methods of using them to treat diseases and conditions.

### Description

Nucleic acid polymers (NAPs) such as REP-2139 (SEQ ID NO: 46) are phosphorothioated oligonucleotides with a repetitive nonspecific sequence that have shown antiviral activity against a range of different viruses, possibly driven by interaction with structurally similar exposed hydrophobic surfaces of amphipathic alpha helices on their targets. The suppression of hepatitis B virus (HBV) and its satellite virus hepatitis delta virus (HDV) are the most impressive: REP-2139 as a monotherapy induced pronounced reductions in HBV surface antigen (HBsAg) titers in chronic hepatitis B (CHB) patients, HBsAg loss, and sustained response off-treatment. Interestingly, NAP therapy in these patients is characterized by a pronounced difference between responders and non-responders. The factors determining response are still unclear. STOPS™ compounds against HBV were developed with significantly improved in vitro properties compared to REP-2139 (U.S. Pat. No. 11,166,976 B2), but did not show sufficient antiviral activity in CHB patients.

### SUMMARY

STOPS™ compounds against HBV have now been developed with improved in vitro potency and in vivo properties in a duck HBV model as compared to clinically studied compound REP-2139. The structures of the new STOPS™ compounds and methods of using them to treat HBV and HBD are surprising and unexpected.

Some embodiments described herein relate to modified oligonucleotides or complexes thereof having sequence independent antiviral activity against HBV, HDV, or both. In some embodiments, the modified oligonucleotides or complexes thereof include a sequence of alternating A and C units having a length of 40 units. In some embodiments, the modified oligonucleotide includes: a 5′ region having 8 units, wherein the A units are selected from 2′-OMe-A and LNA-A, and the C units are selected from 2′-OMe-(5m)C and LNA-(5m)C, wherein 0, 1, 2, 3, or 4 units are independently selected from LNA-A and LNA-(5m)C; a 3′ region having 8 units, wherein the A units are selected from 2′-OMe-A, Ribo-A, and LNA-A, and the C units are selected from 2′-OMe-(5m)C and LNA-(5m)C, wherein 1, 2, 3, or 4 units are independently selected from LNA-A and LNA-(5m)C and 0 or 1 of the A units is Ribo-A; and a central region having 24 units, wherein the A units are selected from 2′-OMe-A and Ribo-A, wherein 0, 1, 2, 3, or 4 units are Ribo-A, and the C units are 2′-OMe-(5m)C.

In some embodiments, all of the A units in the 5′ region and/or the 3′ region are 2′-OMe-A. In some embodiments, all of the A units in the 5′ region are 2′-OMe-A, 3 of the A units in the 3′ region are 2′-OMe-A, and 1 of the A units in the 3′ region is Ribo-A. In some embodiments, the Ribo-A is at position 33 or 35. In some embodiments, 1, 2, 3, or 4 of the C units of the 5′ region are LNA-(5m)C. In some embodiments, 1, 2, or 3 of the C units of the 5′ region and/or 1, 2, or 3 units of the 3′ region are LNA-(5m)C. In some embodiments, 2 of the C units of the 5′ region are LNA-(5m)C and/or 2 of the C units of the 3′ region are LNA-(5m)C. In some embodiments, the C units at position 2, 4, 38, and 40 are LNA-(5m)C. In some embodiments, all of the A units of the central region are 2′-OMe-A. In some embodiments, at least one A unit at position 11, 17, 21, 23, 29, 31, 33, 35, or any combination thereof, is a ribo-A. In some embodiments, 1, 2, or 3 of the A units of the central region are Ribo-A. In some embodiments, all of the A units of the 3′ region are 2′-OMe-A. In some embodiments, the A units at position 11, 21 and 31 are Ribo-A. In some embodiments, the sequence is at least partially phosphorothioated. In some embodiments, the sequence is at least about 85% phosphorothioated. In some embodiments, the sequence is fully phosphorothioated. In some embodiments, the complex is a monovalent counterion complex; preferably wherein the complex includes sodium. In some embodiments, the modified oligonucleotide has an EC₅₀ value, as determined by HBsAg Secretion Assay, less than 100 nM. In some embodiments, the helicity of the oligonucleotide is greater than that SEQ ID NO: 44, as demonstrated by a more sigmoidal Tm curve compared with the Tm curve of SEQ ID NO: 44. In some embodiments, the sequence of alternating A and C units is any one of the sequences of SEQ ID NO: 10-13, 15-21, 23, 25, 27-35, or 37-43. In some embodiments, the sequence of alternating A and C units is any one of the sequences of SEQ ID NO: 38 or 43.

Some embodiments disclosed herein relate to a modified oligonucleotide or complex thereof having sequence independent antiviral activity against HBV, HDV, or both, including a sequence of alternating A and C units, wherein the sequence of alternating A and C units includes SEQ ID NO: 38.

Some embodiments disclosed herein relate to a modified oligonucleotide or complex thereof having sequence independent antiviral activity against HBV, HDV, or both, including a sequence of alternating A and C units, wherein the sequence of alternating A and C units includes SEQ ID NO: 43.

Some embodiments disclosed herein relate to a pharmaceutical composition. In some embodiments, the pharmaceutical composition includes a therapeutic amount of the modified oligonucleotide or complex thereof of any one of the embodiments of the present disclosure, which is effective for treating a subject infected with HBV, HDV, or both; and a pharmaceutically acceptable carrier.

Some embodiments disclosed herein relate to a method of treating hepatitis B, hepatitis D or both, including administering an effective amount of the modified oligonucleotide or complex thereof, or the pharmaceutical composition of any one of the embodiments of the present disclosure, to a subject in need thereof. In some embodiments, the modified oligonucleotide or complex thereof is administered to the subject by a parenteral route. In some embodiments, the modified oligonucleotide or complex thereof is administered to the subject intravenously or subcutaneously. In some embodiments, the methods further include administering an effective amount of a second treatment for hepatitis B, hepatitis D, or both, to the subject. In some embodiments, the second treatment includes a molecule, composition, or treatment known to be effective against hepatitis B, hepatitis D, or both. In some embodiments, the second treatment includes an siRNA oligonucleotide, an anti-sense oligonucleotide, a nucleoside, an interferon, a viral entry inhibitor, an immunomodulator, a capsid assembly modulator, an anti-HBsAg mAb, or a combination thereof. In some embodiments, the second treatment includes administering an ALG-000184, ALG-125755, CAM-E, GSK-836, VIR-2218, recombinant interferon alpha 2b, IFN-gamma, PEG-IFN-alpha-2a, lamivudine, telbivudine, adefovir dipivoxil, clevudine, entecavir, tenofovir alafenamide, tenofovir disoproxil, RG6004, GSK3228836, VIR-3434, BJT-778, Bulevertide, DCR-HBVS, GLS4, NZ-4, and RG7907.

Some embodiments disclosed herein relate to a modified oligonucleotide or complex thereof, wherein the modified oligonucleotide is represented by the following formula: 5′mApsln(5m)CpsmApsln(5m)CpsmApsm(5m)CpsmApsm(5m)CpsmApsm(5m)Cp srApsm(5m)CpsmApsm(5m)CpsmApsm(5m)CpsmApsm(5m)CpsmApsm(5m)CpsrApsm(5 m)CpsmApsm(5m)CpsmApsm(5m)CpsmApsm(5m)CpsmApsm(5m)CpsrApsm(5m)CpsmA psm(5m)CpsmApsm(5m)CpsmApsln(5m)CpsmApsln(5m)C-3′ (SEQ ID NO: 38), wherein mA is 2′-OMe-A, rA is Ribo-A, m (5m)C is 2′-OMe-(5m)C, In (5m)C is LNA-(5m)C, and ps is phosphorothioate.

Some embodiments disclosed herein relate to a modified oligonucleotide or complex thereof, wherein the modified oligonucleotide is represented by the following formula: 5′mApsln(5m)CpsmApsln(5m)CpsmApsm(5m)CpsmApsm(5m)CpsmApsm(5m)Cp smApsm(5m)CpsmApsm(5m)CpsmApsm(5m)CpsmApsm(5m)CpsmApsm(5m)CpsmApsm(5m)CpsmApsm(5m)CpsmApsm(5m)CpsmApsm(5m)CpsmApsm(5m)CpsmApsm(5m)Cpsm Apsm(5m)CpsmApsm(5m)CpsmApsln(5m)CpsmApsln(5m)C-3′ (SEQ ID NO: 43), wherein mA is 2′-OMe-A, m (5m)C is 2′-OMe-(5m)C, ln(5m)C is LNA-(5m)C, and ps is phosphorothioate.

These and other embodiments are described in greater detail below.

### BRIEF DESCRIPTION OF THE DRAWINGS

FIG. 1 illustrates examples of chemical structures corresponding with the abbreviations used in any one of the embodiments of the present disclosure.

FIG. 2A illustrates graphs for the change in helicity (“S” shape of sigmoidal curve) assayed by melting temperature (Tm) analysis of example oligonucleotide structures featuring increasing numbers of LNA units.

FIG. 2B illustrates a graph for the Tm analysis of the sequences of SEQ ID NOs: 38 and 43 compared with reference compounds.

FIGS. 3A-3E illustrate graphs of the reductions from baseline (log scale) for duck hepatitis B virus (DHBV) DNA (left panel) and duck hepatitis B surface antigen (DHBsAg) (right panel) in ducks over 56 days, following 28 days of either no treatment (FIG. 3A), treatment with entecavir (FIG. 3B), treatment with REP-2139 (SEQ ID NO: 46, FIG. 3C), treatment with an oligonucleotide of SEQ ID NO: 44 (FIG. 3D), or treatment with REP2165 (SEQ ID NO: 3, FIG. 3E). Individual ducks are in grey, and the average value is in black.

FIG. 4 illustrates a cartoon schematic of the structures with the sequences of SEQ ID NOs: 38 and 43.

FIGS. 5A-5D illustrate graphs of the reductions from baseline for DHBV DNA (left panel) and DHBsAg (right panel) in ducks over 56 days, following 28 days of either vehicle treatment (FIG. 5A), treatment with REP2139 (SEQ ID NO: 46, FIG. 5B), treatment with a compound of SEQ ID NO: 38 (FIG. 5C), or treatment with a compound of SEQ ID NO: 43 (FIG. 5D). Each plot shows absolute serum titers, wherein individual ducks are in grey, and the median value is in black.

FIGS. 6A-6B illustrate the percent change in response rate for DHBV DNA and DHBsAg at 28 days (left panel) and 56 days (right panel) following treatment for treatment with REP-2139, REP-2165, or SEQ ID NO: 44 (FIG. 6A), or treatment with REP-2139, SEQ ID NO: 38 and 43 (FIG. 6B).

FIG. 7 illustrates embodiments of modified oligonucleotides and corresponding values of sequence independent antiviral activity against HBV (as determined by HBsAg Secretion Assay) and cytotoxicity.

FIG. 8 illustrates a graph for the Tm analysis of oligonucleotides of REP-2139, and SEQ ID NOs: 44 and 45.

### DETAILED DESCRIPTION

The STOPS™ compounds described herein are antiviral oligonucleotides that can be at least partially phosphorothioated and exert their antiviral activity by a non-sequence dependent mode of action. The term “Nucleic Acid Polymer” (NAP) has been used in the literature to refer to such oligonucleotides, although that term does not necessarily connotate antiviral activity. A number of patent applications filed in the early 2000s disclosed the structures of certain specific compounds and identified various structural options as potential areas for future experimentation. See, e.g., U.S. Pat. Nos. 7,358,068; 8,008,269; 8,008,270 and 8,067,385. These efforts resulted in the identification of the compound known to those skilled in the art as REP-2139, a phosphorothioated 40-mer having repeating adenosine-cytidine (AC) units with 5-methylation of all cytosines and 2′-O methyl modification of all riboses. See I. Roehl et al., “Nucleic Acid Polymers with Accelerated Plasma and Tissue Clearance for Chronic Hepatitis B Therapy,” Molecular Therapy: Nucleic Acids Vol. 8, 1-12 (2017). The authors of that publication indicated that the structural features of these compounds had been optimized for the treatment of hepatitis B and hepatitis D. See also A. Vaillant, “Nucleic acid polymers: Broad spectrum antiviral activity, antiviral mechanisms and optimization for the treatment of hepatitis B and hepatitis D infection,” Antiviral Research 133 (2016) 32-40. According to these authors and related literature, such compounds preserve antiviral activity against HBV while preventing recognition by the innate immune response to allow their safe use with immunotherapies such as pegylated interferon. However, there remains a long-felt need for more effective compounds in this class.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

The hepatitis B virus (HBV) is a DNA virus and a member of the Hepadnaviridae family. HBV infects more than 300 million people worldwide and is a causative agent of liver cancer and liver disease such as chronic hepatitis, cirrhosis, and hepatocellular carcinoma. HBV can be acute and/or chronic. Acute HBV infection can be either asymptomatic or present with symptomatic acute hepatitis. HBV is classified into eight genotypes, A to H.

HBV is a partially double-stranded circular DNA of about 3.2 kilobase (kb) pairs. The HBV replication pathway has been studied in great detail. T. J. Liang, Hepatology (2009) 49 (5 Suppl): S13-S21. One part of replication includes the formation of the covalently closed circular (cccDNA) form. The presence of the cccDNA gives rise to the risk of viral reemergence throughout the life of the host organism. HBV carriers can transmit the disease for many years. An estimated 257 million people are living with chronic hepatitis B virus infection, and it is estimated that over 750,000 people worldwide die of hepatitis B each year. In addition, immunosuppressed individuals or individuals undergoing chemotherapy are especially at risk for reactivation of an HBV infection.

HBV can be transmitted by blood, semen, and/or another body fluid. This can occur through direct blood-to-blood contact, unprotected sex, sharing of needles, and from an infected mother to her baby during the delivery process. The HBV surface antigen (HBsAg) is most frequently used to screen for the presence of this infection. Currently available medications do not cure HBV and/or HDV infections. Rather, the medications suppress replication of the virus.

The hepatitis D virus (HDV) is an RNA virus. HDV can propagate only in the presence of HBV. The routes of transmission of HDV are similar to those for HBV. Transmission of HDV can occur either via simultaneous infection with HBV (coinfection) or in addition to chronic hepatitis B or hepatitis B carrier state (superinfection). Both superinfection and coinfection with HDV results in more severe complications compared to infection with HBV alone. These complications include a greater likelihood of experiencing liver failure in acute infections and a rapid progression to liver cirrhosis, with an increased risk of developing liver cancer in chronic infections. In combination with hepatitis B, hepatitis D has the highest fatality rate of all the hepatitis infections, at 20%.

As used herein in the context of oligonucleotides or other materials, the term “antiviral” has its usual meaning as understood by those skilled in the art and thus includes an effect of the presence of the oligonucleotides or other material that inhibits production of viral particles, typically by reducing the number of infectious viral particles formed in a system otherwise suitable for formation of infectious viral particles for at least one virus. In certain embodiments, the antiviral oligonucleotide has antiviral activity against multiple different virus, e.g., both HBV and HDV.

As used herein the term “oligonucleotide” (or “oligo”) has its usual meaning as understood by those skilled in the art and thus refers to a class of compounds that includes oligodeoxynucleotides, oligodeoxyribonucleotides and oligoribonucleotides. Thus, “oligonucleotide” refers to an oligomer or polymer of ribonucleic acid (RNA) or deoxyribonucleic acid (DNA) or mimetics thereof, including reference to oligonucleotides composed of naturally occurring nucleobases, sugars, and phosphodiester (PO) internucleoside (backbone) linkages as well as “modified” or substituted oligonucleotides having non-naturally occurring portions which function similarly. Thus, the term “modified” (or “substituted”) oligonucleotide has its usual meaning as understood by those skilled in the art and includes oligonucleotides having one or more of various modifications, e.g., stabilizing modifications, and thus can include at least one modification in the internucleoside linkage and/or on the ribose, and/or on the base. For example, a modified oligonucleotide can include modifications at the 2′-position of the ribose, acyclic nucleotide analogs, methylation of the base, phosphorothioated (PS) linkages, phosphorodithioate linkages, methylphosphonate linkages, linkages that connect to the sugar ring via sulfur or nitrogen, and/or other modifications as described elsewhere herein. Thus, a modified oligonucleotide can include one or more phosphorothioated (PS) linkages, instead of or in addition to PO linkages. Like unmodified oligonucleotides, modified oligonucleotides that include such PS linkages are considered to be in the same class of compounds because even though the PS linkage contains a phosphorous-sulfur double bond instead of the phosphorous-oxygen double bond of a PO linkage, both PS and PO linkages connect to the sugar rings through oxygen atoms.

As used herein in the context of modified oligonucleotides, the term “phosphorothioated” oligonucleotide has its usual meaning as understood by those skilled in the art and thus refers to a modified oligonucleotide in which all of the phosphodiester internucleoside linkages have been replaced by phosphorothioate linkages. Those skilled in the art thus understand that the term “phosphorothioated” oligonucleotide is synonymous with “fully phosphorothioated” oligonucleotide. A phosphorothioated oligonucleotide (or a sequence of phosphorothioated oligonucleotides within a partially phosphorothioated oligonucleotide) can be modified analogously, including (for example) by replacing one or more phosphorothioated internucleoside linkages by phosphodiester linkages. Thus, the term “modified phosphorothioated” oligonucleotide refers to a phosphorothioated oligonucleotide that has been modified in the manner analogous to that described herein with respect to oligonucleotides, e.g., by replacing a phosphorothioated linkage with a modified linkage such as phosphodiester, phosphorodithioate, methylphosphonate, diphosphorothioate, 5′-phosphoramidate, 3′,5′-phosphordiamidate, 5′-thiophosphoramidate, 3′,5′-thiophosphordiamidate or diphosphodiester. An at least partially phosphorothioated sequence of a modified oligonucleotide can be modified similarly, and thus, for example, can be modified to contain a non-phosphorothioated linkage such as phosphodiester, phosphorodithioate, methylphosphonate, diphosphorothioate 5′-phosphoramidate, 3′,5′-phosphordiamidate, 5′-thiophosphoramidate, 3′,5′-thiophosphordiamidate or diphosphodiester. In the context of describing modifications to a phosphorothioated oligonucleotide, or to an at least partially phosphorothioated sequence of a modified oligonucleotide, modification by inclusion of a phosphodiester linkage may be considered to result in a modified phosphorothioated oligonucleotide, or to a modified phosphorothioated sequence, respectively. Analogously, in the context of describing modifications to an oligonucleotide, or to an at least partially phosphodiesterified sequence of a modified oligonucleotide, the inclusion of a phosphorothioated linkage may be considered to result in a modified oligonucleotide or a modified phosphodiesterified sequence, respectively.

As used herein in the context of dinucleotides or oligonucleotides, the term “stereochemically defined phosphorothioate linkage” has its usual meaning as understood by those skilled in the art and thus refers to a phosphorothioate linkage having a phosphorus stereocenter with a selected chirality (R or S configuration). A composition containing such a dinucleotide or oligonucleotide can be enriched in molecules having the selected chirality. The stereopurity of such a composition can vary over a broad range, e.g. from about 51% to about 100% stereopure. In various embodiments, the stereopurity is greater than 55%, 65%, 75%, 85%, 90%, 91%, 92%, 93%, 94%, 95%, 96%, 97%, 98%, or 99%; or in a range defined as having any two of the foregoing stereopurity values as endpoints.

The term “sequence independent” antiviral activity has its usual meaning as understood by those skilled in the art and thus refers to an antiviral activity of an oligonucleotide (e.g., a modified oligonucleotide) that is independent of the sequence of the oligonucleotide. Methods for determining whether the antiviral activity of an oligonucleotide is sequence independent are known to those skilled in the art and include the tests for determining if an oligonucleotide acts predominantly by a non-sequence complementary mode of action as disclosed in Example 10 of U.S. Pat. Nos. 7,358,068; 8,008,269; 8,008,270 and 8,067,385, which is hereby incorporated herein by reference and particularly for the purpose of describing such tests.

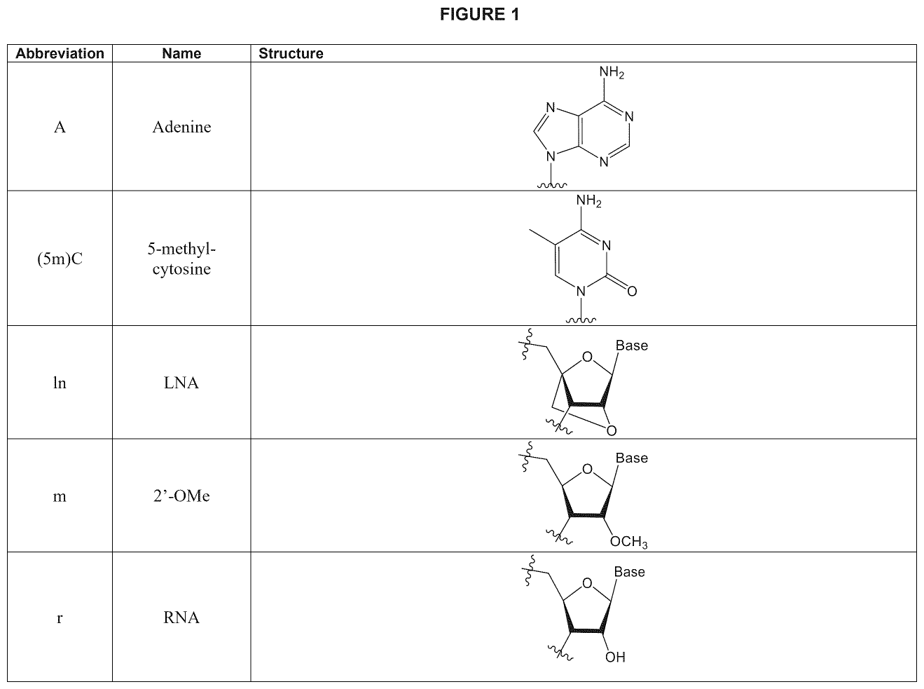

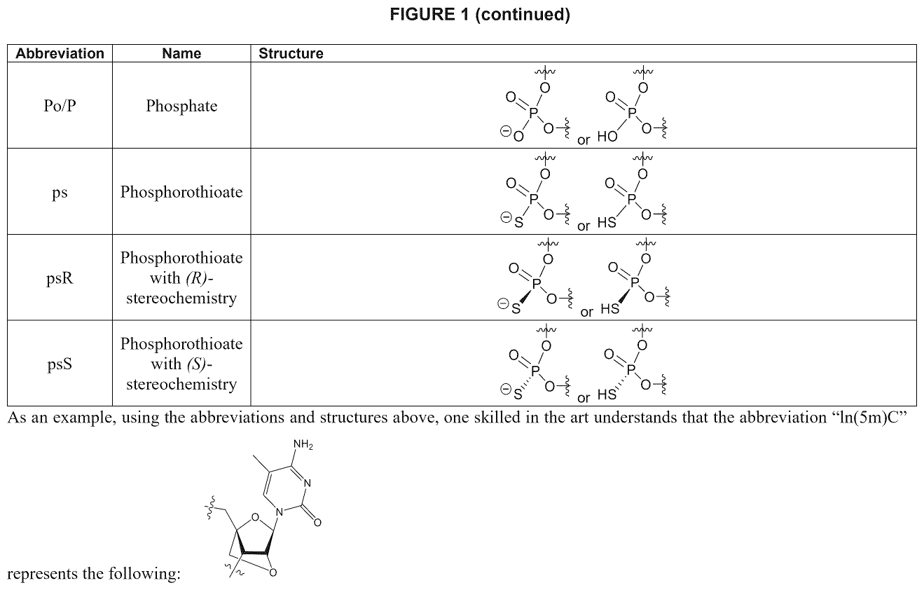

In the context of describing modified oligonucleotides having sequence independent antiviral activity and including a sequence (e.g., an at least partially phosphorothioated sequence) of A and C units (e.g., alternating A and C units, or AC units), the terms “A” and “C” refer to the modified adenosine-containing (A) units and modified cytidine-containing (C) units set forth in FIG. 1 and in Tables 1 and 2 below, respectively.

TABLE 1
“A” UNITS

| Abbreviation (A Unit) | Structure (A Unit) |
|---|---|
| 2′-OMe-A | 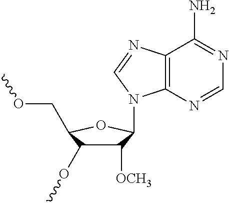 |
| LNA-A | 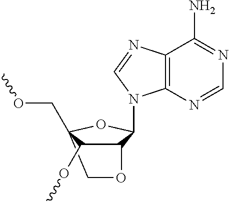 |
| Ribo-A | 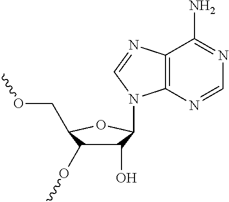 |

TABLE 2
“C” UNITS

| Abbreviation (C Unit) | Structure (C Unit) |
|---|---|
| 2′-OMe-(5m)C | 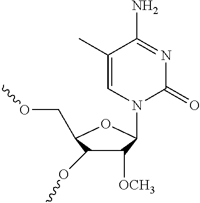 |
| LNA-(5m)C | 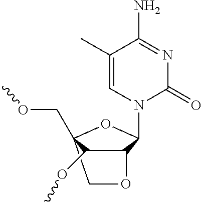 |

### Testing of Modified Oligonucleotide Compounds

Nucleic acid polymers (NAPs) such as REP-2139 are phosphorothioated oligonucleotides with a repetitive nonspecific sequence that have shown antiviral activity against a range of different viruses, possibly driven by interaction with structurally similar exposed hydrophobic surfaces of amphipathic alpha helices on their targets. The suppression of hepatitis B virus (HBV) and its satellite virus hepatitis delta virus (HDV) are the most impressive: REP2139 as a monotherapy induced pronounced reductions in HBV surface antigen (HBsAg) titers in 75% of (9/12) chronic hepatitis B (CHB) patients, HBsAg loss in 25% (3/12), and sustained response off-treatment in 17% (2/12). Combination with pegylated interferon (PEG-IFNα-2a) further increased functional cure rates to 35% (14/40). REP-2139 also reduced HDV titers below detection limits with off-treatment cure rates as high as 64% (7/11). Interestingly, NAP therapy in these patients is characterized by a pronounced difference between responders and non-responders. The factors determining response are still unclear.

Several efforts to understand the underlying mechanism of action of NAPs have been reported: inhibition of HBsAg secretion without intracellular accumulation was shown in vitro, interactions with several host factors were demonstrated, and parallels with lipoprotein metabolism have been drawn. Nevertheless, no single mechanism could be definitively confirmed. Even more intriguingly, the pronounced clinical efficacy of NAPs could not be reproduced in commonly used mouse models of CHB or in the related woodchuck model. The only model that has shown some promise at recapitulating the clinical efficacy of NAPs so far has been the duck HBV (DHBV) model. Earlier studies showed a response in most DHBV-infected ducks treated with intraperitoneally (IP) administered REP-2139 and explored combination therapy with nucleoside analogs (NUCs) commonly used for chronic viral suppression in CHB patients. Although these studies generated important insights, they also highlighted the need for further improvements to the model (e.g. high drop-out rates) and its read-outs (often qualitative), and left many questions unanswered.

The DHBV model has been a critical tool for the early understanding of many key fundamental aspects of HBV and hepadnaviral biology, particularly the role and functioning of the covalently closed circular DNA (cccDNA) within the viral life cycle. DHBV has a similar genome structure as human HBV, with some notable differences such as the presence of a cryptic HBx transcription initiation site and the lack of a middle HBsAg. In recent years, use of the DHBV duck model has become less common due to its challenging nature (for example, the limited availability of duck-specific reagents, lack of inbred duck strains, and/or specific maintenance requirements) and the advent of more convenient models such as the adeno-associated virus (AAV) HBV mouse model, even though these are unable to capture all relevant aspects of the HBV replication cycle and its associated immune responses.

CHB, with or without HDV co-infection, still represents an important medical need and no other therapy has shown such promising response rates as NAPs have. A better understanding of NAP efficacy and their underlying mechanism of action requires a reliable, optimized, and translatable animal model. As disclosed herein, the DHBV duck model recapitulates essential aspects of the clinical response to NAPs in CHB patients.

### Modified Oligonucleotide Compounds

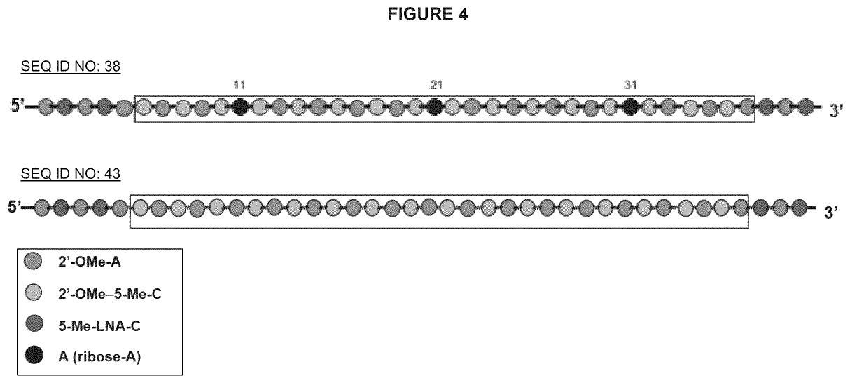

Some embodiments provided herein relate to STOPS™ modified oligonucleotide compound having sequence independent antiviral activity against HBV, including an at least partially phosphorothioated sequence of alternating A and C units, wherein the A units are any one or more selected from those set forth in Table 1 and the C units are any one or more selected from those set forth in Table 2. Various combinations of A and C units can be included in the at least partially phosphorothioated AC sequence, including any combination of ln(5m)C (LNA-(5m)C), lnA (LNA-A), mA (2′-OMe-A), m(5m)C (2′-OMe-(5m)C), and rA (Ribo-A) units. In some embodiments, the combination of A and C units is as shown in any embodiment of FIGS. 4 and/or 7.

As described elsewhere herein, a modified oligonucleotide can include a single at least partially phosphorothioated sequence of alternating A and C units. In some embodiments, the modified oligonucleotide can include a plurality of at least partially phosphorothioated sequences of alternating A and C units that are linked together. Thus, a modified oligonucleotide that contains a single at least partially phosphorothioated sequence of alternating A and C units can have the same sequence length as that sequence. Examples of such sequence lengths are described elsewhere herein. Similarly, a modified oligonucleotide that contains a plurality of at least partially phosphorothioated sequences of alternating A and C units can have sequence length that is the result of linking those sequences as described elsewhere herein. Examples of sequence lengths for a modified oligonucleotide that contains a plurality of at least partially phosphorothioated sequences of alternating A and C units are expressed elsewhere herein in terms of the lengths of the individual sequences, and also taking into account the length of the linking group.

Some embodiments disclosed herein relate to a modified oligonucleotide or complex thereof having sequence independent antiviral activity against HBV, HDV, or both, including a sequence of alternating A and C units having a length of 40 units. In some embodiments, the modified oligonucleotide includes a 5′ region including 8 units, wherein the A units are selected from 2′-OMe-A and LNA-A, the C units are selected from 2′-OMe-(5m)C and LNA-(5m)C, and wherein 0, 1, 2, 3, or 4 units are independently selected from LNA-A and LNA-(5m)C. In some embodiments, the modified oligonucleotide includes a 3′ region including 8 units, wherein the A units are selected from 2′-OMe-A, Ribo-A and LNA-A, the C units are selected from 2′-OMe-(5m)C and LNA-(5m)C, and wherein 1, 2, 3 or 4 units are independently selected from LNA-A and LNA-(5m)C and 0 or 1 of the A units is Ribo-A. In some embodiments, the modified oligonucleotide includes a central region including 24 units, wherein the A units are selected from 2′-OMe-A and Ribo-A, wherein 0, 1, 2, 3, or 4 units are Ribo-A, and wherein the C units are 2′-OMe-(5m)C. In some embodiments, all of the A units in the 5′ region and/or the 3′ region are 2′-OMe-A. In some embodiments, all of the A units in the 5′ region are 2′-OMe-A, 3 of the units in the 3′ region are 2′-OMe-A, and 1 of the units in the 3′ region is Ribo-A. In some embodiments, 1, 2, 3, or 4 units of the 5′ region are LNA-(5m)C. In some embodiments, 1, 2, or 3 units of the 5′ region and/or 1, 2, or 3 units of the 3′ region are LNA-(5m)C. In some embodiments, 2 units of the 5′ region are LNA-(5m)C and/or 2 units of the 3′ region are LNA-(5m)C. In some embodiments, the C units at position 2, 4, 38 and 40 are LNA-(5m)C. In some embodiments, all of the A units of the central region are 2′OMe-A. In some embodiments, when an A unit is ribo-A, the ribo-A is at position 11, 17, 21, 23, 29, 31, 33, 35, or any combination thereof from 5′ end of sequence. In some embodiments, 1, 2, or 3 of the A units of the central region are Ribo-A, wherein the ribo-A is at position 11, 17, 21, 23, 29, 31, or any combination thereof. In some embodiments, 3 of the A units are ribo-A, wherein the ribo-A units are at position 11, 21 and 31. In some embodiments, the sequence is at least partially phosphorothioated; preferably wherein the sequence is at least about 85% phosphorothioated. In some embodiments, the sequence is fully phosphorothioated. In some embodiments, the modified oligonucleotide or complex thereof includes an at least one R or S configured phosphorothioate linkage. In some embodiments, the modified oligonucleotide or complex thereof further includes an at least one second oligonucleotide that is attached to the modified oligonucleotide via a linking group. In some embodiments, the complex is a monovalent counterion complex; preferably wherein the complex includes sodium. In some embodiments, the modified oligonucleotide has an EC₅₀ value, as determined by HBsAg Secretion Assay, less than 100 nM. In some embodiments, the helicity of the oligonucleotide is greater than that of SEQ ID NO: 44. In some embodiments, the sequence of alternating A and C units is any one of the sequences of SEQ ID NOs: 10-13, 15-21, 23, 25, 27-35, or 37-43. In some embodiments, the sequence of alternating A and C units is any one of the sequences of SEQ ID NOs: 38 or 43. In some embodiments, fewer LNA units results in increased helicity, wherein increased helicity means that the structure of the oligonucleotide is able to form a more helical structure. Helicity can be determined by measuring a Tm curve, wherein the Tm curve becomes more sigmoidal as the LNA units are reduced.

In various embodiments, the at least partially phosphorothioated sequence of alternating A and C units can include modification(s) to one or more phosphorothioated linkages. The inclusion of such a modified linkage is not ordinarily considered to interrupt the alternating sequence of A and C units because those skilled in the art understand that such a sequence may be only partially phosphorothioated and thus may include one or more modifications to a phosphorothioate linkage. In various embodiments, the modification to the phosphorothioate linkage is a modified linkage selected from phosphodiester, phosphorodithioate, methylphosphonate, diphosphorothioate and diphosphodiester. For example, in some embodiments, the modified linkage is a phosphodiester linkage.

In various embodiments, the at least partially phosphorothioated sequence of alternating A and C units can have various degrees of phosphorothioation. For example, in some embodiments, the at least partially phosphorothioated sequence of alternating A and C units is at least 50%, 55%, 60%, 65%, 70%, 75%, 80%, 85%, 90%, or 95% phosphorothioated. In some embodiments, the at least partially phosphorothioated sequence of alternating A and C units is at least 85% phosphorothioated. In some embodiments, the at least partially phosphorothioated sequence of alternating A and C units is fully phosphorothioated.

In various embodiments, the at least partially phosphorothioated sequence of alternating A and C units can include stereochemical modification(s) to one or more phosphorothioated linkages. In some embodiments, the modified oligonucleotides described herein can include at least one stereochemically defined phosphorothioate linkage. In various embodiments, the stereochemically defined phosphorothioate linkage has an R configuration. In various embodiments, the stereochemically defined phosphorothioate linkage has an S configuration.

Those skilled in the art will recognize that modified oligonucleotide compounds including A and C units as described herein, such as the A and C units of Tables 1 and 2, respectively, contain internal linkages between the A and C units as well as terminal groups at the 3′ and 5′ ends. Thus, with respect to the A and C units described herein, such as the A and C units of Tables 1 and 2, respectively, each [symbol] represents an internal [symbol] or a terminal [symbol] . In various embodiments, each terminal [symbol] is independently hydroxyl, an O,O-dihydrogen phosphorothioate, a dihydrogen phosphate, an endcap, or a linking group. In various embodiments, each internal [symbol] is a phosphorus-containing linkage to a neighboring A or C unit, the phosphorus-containing linkage being a phosphorothioate linkage or a modified linkage selected from phosphodiester, phosphorodithioate, methylphosphonate, diphosphorothioate 5′-phosphoramidate, 3′,5′-phosphordiamidate, 5′-thiophosphoramidate, 3′,5′-thiophosphordiamidate or diphosphodiester.

In various embodiments, a modified oligonucleotide as described herein, including an at least partially phosphorothioated sequence of alternating A and C units, has sequence independent antiviral activity against HBV, as determined by HBsAg Secretion Assay, that is in an “A” activity range of less than 30 nanomolar (nM); in a “B” activity range of 30 nM to less than 100 nM; in a “C” activity range of 100 nM to less than 300 nM; or in a “D” activity range of greater than 300 nM. In some embodiments, a modified oligonucleotide as described herein, including an at least partially phosphorothioated sequence of alternating A and C units, has sequence independent antiviral activity against HBV, as determined by HBsAg Secretion Assay, less than 30 nM.

The modified oligonucleotides described herein can be prepared in the form of various complexes. Thus, some embodiments provide a chelate complex of a modified oligonucleotide as described herein, such as monovalent counterion complexes. For example, in some embodiments such a counterion complex includes a lithium, sodium or potassium complex of the modified oligonucleotide.

### Synthesis

The modified oligonucleotides described herein can be prepared in various ways. In some embodiments, the building block monomers described in Tables 3 and 4 are employed to make the modified oligonucleotides described herein by applying standard phosphoramidite chemistry. The building blocks described in Tables 3 and 4 and other building block phosphoramidite monomers can be prepared by known methods or obtained from commercial sources (Thermo Fischer Scientific US, Hongene Biotechnology USA Inc., Chemgenes Corporation). Exemplary procedures for making modified oligonucleotides are set forth in the Examples below.

TABLE 3
BUILDING BLOCKS FOR “A” UNITS

| Abbreviation | Structure |
|---|---|
| 2′-OMe-A PHOSPHORAMIDITE | 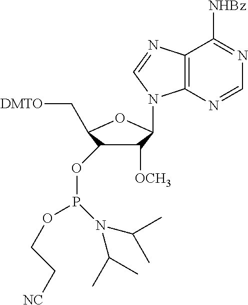 |
| LNA-A PHOSPHORAMIDITE | 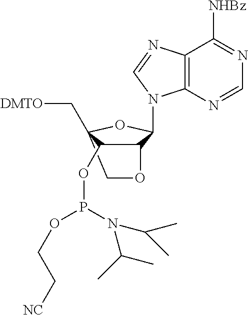 |
| Ribo-A PHOSPHORAMIDITE | 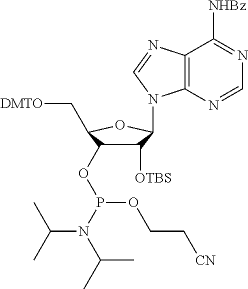 |

TABLE 4
BUILDING BLOCKS FOR “C” UNITS

| Abbreviation | Structure |
|---|---|
| 2′-OMe-(5m)C PHOSPHORAMIDITE | 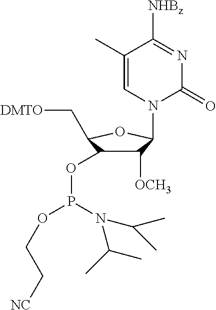 |
| LNA-(5m)C PHOSPHORAMIDITE | 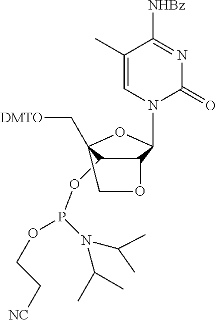 |

In various embodiments, the STOPS™ modified oligonucleotides described herein can also be prepared using dinucleotides that includes or consists of any two of the building block monomers described in Tables 3 and 4. Exemplary procedures for making dinucleotides and the corresponding modified oligonucleotides are set forth in the Examples below.

Some embodiments provided herein relate to a dinucleotide including, or consisting of, an A unit and a C unit connected by a stereochemically defined phosphorothioate linkage, wherein the A unit is selected from any of the building block monomers described in Table 3 and the C unit is selected from any of the building block monomers described in Table 4, and wherein each [symbol] is independently hydroxyl, an O,O-dihydrogen phosphorothioate, an O,O-dihydrogen phosphate, a phosphoramidite, a dimethoxytrityl ether, or the stereochemically defined phosphorothioate linkage. In some embodiments, the [symbol] is a phosphoramidite of the following formula (A):

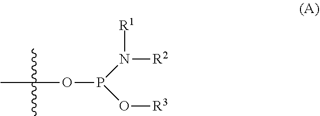

In various embodiments R¹ and R² of formula (A) are each individually a C₁₋₆alkyl, and R³ is a C₁₋₆alkyl or a cyanoC₁₋₆alkyl. For example, in some embodiments, the phosphoramidite of the formula (A) is a phosphoramidite of the following formula (A1):

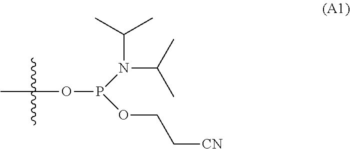 [SMILES: *OP(OCCC#N)N(C(C)C)C(C)C]

In some embodiments, the [symbol] is a stereochemically defined phosphorothioate linkage that is a phosphorothioate. For example, in some embodiments, the stereochemically defined phosphorothioate linkage is a phosphorothioate of the following Formula (B1) or (B2):

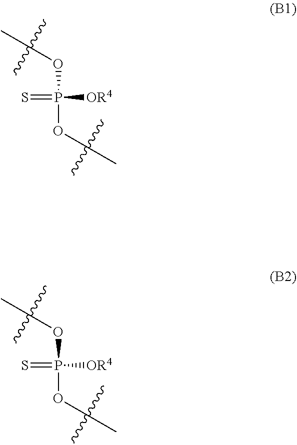

In various embodiments R⁴ of formulae (B1) and (B2) is a C₁₋₆ alkyl or a cyanoC₁₋₆ alkyl. For example, in some embodiments, the phosphorothioates of the formulae (B1) and (B2) are phosphorothioates of the following Formulae (B3) or (B4), respectively:

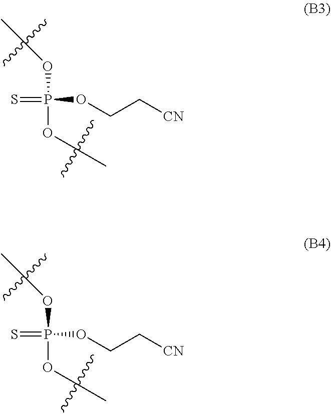

Various embodiments provide methods of making a modified oligonucleotide as described herein, including coupling one or more dinucleotides as described herein. Exemplary methods of carrying out such coupling are illustrated in the Examples below.

### Pharmaceutical Compositions

Some embodiments described herein relate to a pharmaceutical composition, that can include an effective amount of a compound described herein (e.g., a STOPS™ modified oligonucleotide compound or complex thereof as described herein) and a pharmaceutically acceptable carrier, excipient, or combination thereof. Pharmaceutical compositions described herein are suitable for human and/or veterinary applications.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion, or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

Proper formulation is dependent upon the route of administration chosen. Techniques for formulation and administration of the compounds described herein are known to those skilled in the art. Multiple techniques of administering a compound exist in the art including, but not limited to, oral, rectal, topical, aerosol, injection, and parenteral delivery, including intramuscular, subcutaneous, intravenous, intramedullary injections, intrathecal, direct intraventricular, intraperitoneal, intranasal, and intraocular injections. Pharmaceutical compositions will generally be tailored to the specific intended route of administration.

One may also administer the compound in a local rather than systemic manner, for example, via injection of the compound directly into the infected area, optionally in a depot or sustained release formulation. Furthermore, one may administer the compound in a targeted drug delivery system, for example, in a liposome coated with a tissue-specific antibody. The liposomes may be targeted to and taken up selectively by the organ.

The pharmaceutical compositions disclosed herein may be manufactured in a manner that is itself known, e.g., by means of conventional mixing, dissolving, granulating, dragee-making, levigating, emulsifying, encapsulating, entrapping, or tableting processes. As described herein, compounds used in a pharmaceutical composition may be provided as salts with pharmaceutically compatible counterions.

### Methods of Use

Some embodiments described herein relate to a method of treating an HBV and/or HDV infection that can include administering to a subject identified as suffering from the HBV and/or HDV infection an effective amount of a modified oligonucleotide or complex thereof as described herein, or a pharmaceutical composition that includes an effective amount of a modified oligonucleotide or complex thereof as described herein. Some embodiments described herein relate to using a modified oligonucleotide or complex thereof as described herein in the manufacture of a medicament for treating an HBV and/or HDV infection. Some embodiments described herein relate to the use of a modified oligonucleotide or complex thereof as described herein or a pharmaceutical composition that includes a modified oligonucleotide as described herein for treating an HBV and/or HDV infection.

Various routes may be used to administer a modified oligonucleotide or complex thereof to a subject in need thereof as indicated elsewhere herein. In some embodiments, the modified oligonucleotide or complex thereof is administered to the subject by a parenteral route. For example, in some embodiments, the modified oligonucleotide or complex thereof is administered to the subject intravenously. In some embodiments, the modified oligonucleotide or complex thereof is administered to the subject subcutaneously.

Some embodiments disclosed herein relate to a method of treating an HBV and/or HDV infection that can include contacting a cell infected with the HBV and/or HDV with an effective amount of a modified oligonucleotide or complex thereof as described herein, or a pharmaceutical composition that includes an effective amount of a modified oligonucleotide or complex thereof as described herein. In some embodiments, such a method of treating an HBV and/or HDV infection includes safe and effective subcutaneous administration of the modified oligonucleotide or complex thereof to a human. In some embodiments, the modified oligonucleotide or complex thereof includes a highly potent STOPS™ compound or complex thereof as described herein. For example, in some embodiments, the STOPS™ compound or complex thereof is a modified oligonucleotide or complex thereof as described herein, including an at least partially phosphorothioated sequence of alternating A and C units, having sequence independent antiviral activity against HBV, as determined by HBsAg Secretion Assay, which is in an “A” activity range of less than 30 nM. In some embodiments, the modified oligonucleotide or complex thereof is SEQ ID NO: 38 or SEQ ID NO: 43.

Some embodiments described herein relate to using a modified oligonucleotide or complex thereof as described herein in the manufacture of a medicament for treating an HBV and/or HDV infection. Some embodiments described herein relate to the use of a modified oligonucleotide or complex thereof as described herein, or a pharmaceutical composition that includes an effective amount of a modified oligonucleotide or complex thereof as described herein for treating an HBV and/or HDV infection. In some embodiments, such uses include safe and effective subcutaneous administration of the modified oligonucleotide or complex thereof to a human. In some embodiments, the modified oligonucleotide or complex thereof includes a highly potent STOPS™ compound or complex thereof as described herein. For example, in some embodiments, the STOPS™ compound or complex thereof is a modified oligonucleotide or complex thereof as described herein, including an at least partially phosphorothioated sequence of alternating A and C units, having sequence independent antiviral activity against HBV, as determined by HBsAg Secretion Assay, which is in an “A” activity range of less than 30 nM. In some embodiments, the modified oligonucleotide or complex thereof is SEQ ID NO: 38 or SEQ ID NO: 43.

Some embodiments disclosed herein relate to a method of inhibiting replication of HBV and/or HDV that can include contacting a cell infected with the HBV and/or HDV with an effective amount of a modified oligonucleotide or complex thereof as described herein, or a pharmaceutical composition that includes an effective amount of a modified oligonucleotide or complex thereof as described herein. In some embodiments, such a method of inhibiting replication of HBV and/or HDV includes safe and effective subcutaneous administration of the modified oligonucleotide or complex thereof to a human. In some embodiments, the modified oligonucleotide or complex thereof includes a highly potent STOPS™ compound or complex thereof as described herein. For example, in some embodiments, the STOPS™ compound or complex thereof is a modified oligonucleotide or complex thereof as described herein, including an at least partially phosphorothioated sequence of alternating A and C units, having sequence independent antiviral activity against HBV, as determined by HBsAg Secretion Assay, which is in an “A” activity range of less than 30 nM. In some embodiments, the modified oligonucleotide or complex thereof is SEQ ID NO: 38 or SEQ ID NO: 43.

Some embodiments described herein relate to using a modified oligonucleotide or complex thereof as described herein in the manufacture of a medicament for inhibiting replication of HBV and/or HDV. Some embodiments described herein relate to the use of a modified oligonucleotide or complex thereof as described herein, or a pharmaceutical composition that includes an effective amount of a modified oligonucleotide or complex thereof as described herein, for inhibiting replication of HBV and/or HDV. In some embodiments, such uses for inhibiting replication of HBV and/or HDV include safe and effective subcutaneous administration of the modified oligonucleotide or complex thereof to a human. In some embodiments, the modified oligonucleotide or complex thereof includes a highly potent STOPS™ compound or complex thereof as described herein. For example, in some embodiments, the STOPS™ compound or complex thereof is a modified oligonucleotide or complex thereof as described herein, including an at least partially phosphorothioated sequence of alternating A and C units, having sequence independent antiviral activity against HBV, as determined by HBsAg Secretion Assay, which is in an “A” activity range of less than 30 nM. In some embodiments, the modified oligonucleotide or complex thereof is SEQ ID NO: 38 or SEQ ID NO: 43.

In some embodiments, the HBV infection can be an acute HBV infection. In some embodiments, the HBV infection can be a chronic HBV infection.

Some embodiments disclosed herein relate to a method of treating liver cirrhosis that is developed because of a HBV and/or HDV infection that can include administering to a subject suffering from liver cirrhosis and/or contacting a cell infected with the HBV and/or HDV in a subject suffering from liver cirrhosis with an effective amount of a modified oligonucleotide or complex thereof as described herein, or a pharmaceutical composition that includes an effective amount of a modified oligonucleotide or complex thereof as described herein. In some embodiments, such a method of treating liver cirrhosis that is developed because of an HBV and/or HDV infection includes safe and effective subcutaneous administration of the modified oligonucleotide or complex thereof to a human. In some embodiments, the modified oligonucleotide or complex thereof includes a highly potent STOPS™ compound or complex thereof as described herein. For example, in some embodiments, the STOPS™ compound or complex thereof is a modified oligonucleotide or complex thereof as described herein, including an at least partially phosphorothioated sequence of alternating A and C units, having sequence independent antiviral activity against HBV, as determined by HBsAg Secretion Assay, which is in an “A” activity range of less than 30 nM. In some embodiments, the modified oligonucleotide or complex thereof is SEQ ID NO: 38 or SEQ ID NO: 43.

Some embodiments described herein relate to using a modified oligonucleotide or complex thereof as described herein in the manufacture of a medicament for treating liver cirrhosis that is developed because of an HBV and/or HDV infection, with an effective amount of the modified oligonucleotide(s). Some embodiments described herein relate to the use of a modified oligonucleotide or complex thereof as described herein, or a pharmaceutical composition that includes an effective amount of a modified oligonucleotide or complex thereof as described herein for treating liver cirrhosis that is developed because of an HBV and/or HDV infection. In some embodiments, such uses for treating liver cirrhosis include safe and effective subcutaneous administration of the modified oligonucleotide or complex thereof to a human. In some embodiments, the modified oligonucleotide or complex thereof includes a highly potent STOPS™ compound or complex thereof as described herein. For example, in some embodiments, the STOPS™ compound or complex thereof is a modified oligonucleotide or complex thereof as described herein, including an at least partially phosphorothioated sequence of alternating A and C units, having sequence independent antiviral activity against HBV, as determined by HBsAg Secretion Assay, which is in an “A” activity range of less than 30 nM. In some embodiments, the modified oligonucleotide or complex thereof is SEQ ID NO: 38 or SEQ ID NO: 43.

Some embodiments disclosed herein relate to a method of treating liver cancer (such as hepatocellular carcinoma) that has developed because of HBV and/or HDV infection that can include administering to a subject suffering from the liver cancer and/or contacting a cell infected with the HBV and/or HDV in a subject suffering from the liver cancer with an effective amount of a modified oligonucleotide or complex thereof as described herein, or a pharmaceutical composition that includes an effective amount of a modified oligonucleotide or complex thereof as described herein. In some embodiments, such a method of treating liver cancer (such as hepatocellular carcinoma) that has developed because of HBV and/or HDV infection includes safe and effective subcutaneous administration of the modified oligonucleotide or complex thereof to a human. In some embodiments, the modified oligonucleotide or complex thereof includes a highly potent STOPS™ compound or complex thereof as described herein. For example, in some embodiments, the STOPS™ compound or complex thereof is a modified oligonucleotide or complex thereof as described herein, including an at least partially phosphorothioated sequence of alternating A and C units, having sequence independent antiviral activity against HBV, as determined by HBsAg Secretion Assay, which is in an “A” activity range of less than 30 nM. In some embodiments, the modified oligonucleotide or complex thereof is SEQ ID NO: 38 or SEQ ID NO: 43.

Some embodiments described herein relate to using a modified oligonucleotide or complex thereof as described herein in the manufacture of a medicament for treating liver cancer (such as hepatocellular carcinoma) that has developed because of HBV and/or HDV infection. Some embodiments described herein relate to the use of a modified oligonucleotide or complex thereof as described herein, or a pharmaceutical composition that includes an effective amount of a modified oligonucleotide or complex thereof as described herein for treating liver cancer (such as hepatocellular carcinoma) that has developed because of HBV and/or HDV infection. In some embodiments, such uses for treating liver cancer (such as hepatocellular carcinoma) include safe and effective subcutaneous administration of the modified oligonucleotide or complex thereof to a human. In some embodiments, the modified oligonucleotide or complex thereof includes a highly potent STOPS™ compound or complex thereof as described herein. For example, in some embodiments, the STOPS™ compound or complex thereof is a modified oligonucleotide or complex thereof as described herein, including an at least partially phosphorothioated sequence of alternating A and C units, having sequence independent antiviral activity against HBV, as determined by HBsAg Secretion Assay, which is in an “A” activity range of less than 30 nM. In some embodiments, the modified oligonucleotide or complex thereof is SEQ ID NO: 38 or SEQ ID NO: 43.

Some embodiments disclosed herein relate to a method of treating liver failure that has developed because of HBV and/or HDV infection that can include administering to a subject suffering from liver failure and/or contacting a cell infected with the HBV and/or HDV in a subject suffering from liver failure with an effective amount of a modified oligonucleotide or complex thereof as described herein, or a pharmaceutical composition that includes an effective amount of a modified oligonucleotide or complex thereof as described herein. In some embodiments, such a method of treating liver failure that is has developed because of HBV and/or HDV infection includes safe and effective subcutaneous administration of the modified oligonucleotide or complex thereof to a human. In some embodiments, the modified oligonucleotide or complex thereof includes a highly potent STOPS™ compound or complex thereof as described herein. For example, in some embodiments, the STOPS™ compound or complex thereof is a modified oligonucleotide or complex thereof as described herein, including an at least partially phosphorothioated sequence of alternating A and C units, having sequence independent antiviral activity against HBV, as determined by HBsAg Secretion Assay, which is in an “A” activity range of less than 30 nM. In some embodiments, the modified oligonucleotide or complex thereof is SEQ ID NO: 38 or SEQ ID NO: 43.

Some embodiments described herein relate to using a modified oligonucleotide or complex thereof as described herein in the manufacture of a medicament for treating liver failure that has developed because of HBV and/or HDV infection. Some embodiments described herein relate to the use of a modified oligonucleotide or complex thereof as described herein, or a pharmaceutical composition that includes an effective amount of a modified oligonucleotide or complex thereof as described herein for treating liver failure that has developed because of HBV and/or HDV infection. In some embodiments, such uses for treating liver failure include safe and effective subcutaneous administration of the modified oligonucleotide or complex thereof to a human. In some embodiments, the modified oligonucleotide or complex thereof includes a highly potent STOPS™ compound or complex thereof as described herein. For example, in some embodiments, the STOPS™ compound or complex thereof is a modified oligonucleotide or complex thereof as described herein, including an at least partially phosphorothioated sequence of alternating A and C units, having sequence independent antiviral activity against HBV, as determined by HBsAg Secretion Assay, which is in an “A” activity range of less than 30 nM. In some embodiments, the modified oligonucleotide or complex thereof is SEQ ID NO: 38 or SEQ ID NO: 43.

Various indicators for determining the effectiveness of a method for treating an HBV and/or HDV infection are also known to those skilled in the art. Examples of suitable indicators include, but are not limited to, a reduction in viral load indicated by reduction in HBV DNA (or load), HBV surface antigen (HBsAg) and HBV e-antigen (HBeAg), a reduction in plasma viral load, a reduction in viral replication, a reduction in time to seroconversion (virus undetectable in patient serum), an increase in the rate of sustained viral response to therapy, an improvement in hepatic function, and/or a reduction of morbidity or mortality in clinical outcomes.

In some embodiments, an effective amount of a modified oligonucleotide or complex thereof as described herein is an amount that is effective to achieve a sustained virologic response, for example, a sustained viral response 12 month after completion of treatment.

Subjects who are clinically diagnosed with an HBV and/or HDV infection include “naïve” subjects (e.g., subjects not previously treated for hepatitis B and/or hepatitis D) and subjects who have failed prior treatment for hepatitis B and/or hepatitis D (“treatment failure” subjects). Treatment failure subjects include “non-responders” (subjects who did not achieve sufficient reduction in ALT levels, for example, subject who failed to achieve more than 1 log 10 decrease from base-line within 6 months of starting an anti-HBV and/or anti-HDV therapy) and “relapsers” (subjects who were previously treated for hepatitis B and/or hepatitis D whose ALT levels have increased, for example, ALT is more than twice the upper normal limit and detectable serum HBV DNA by hybridization assays). Further examples of subjects include subjects with an HBV and/or HDV infection who are asymptomatic.

In some embodiments, a modified oligonucleotide or complex thereof as described herein can be provided to a treatment failure subject suffering from hepatitis B and/or hepatitis D. In some embodiments, a modified oligonucleotide or complex thereof as described herein can be provided to a non-responder subject suffering from hepatitis B and/or hepatitis D. In some embodiments, a modified oligonucleotide or complex thereof as described herein can be provided to a relapser subject suffering from hepatitis B and/or hepatitis D. In some embodiments, the subject can have HBeAg positive chronic hepatitis B. In some embodiments, the subject can have HBeAg negative chronic hepatitis B. In some embodiments, the subject can have liver cirrhosis. In some embodiments, the subject can be asymptomatic, for example, the subject can be infected with HBV and/or HDV but does not exhibit any symptoms of the viral infection. In some embodiments, the subject can be immunocompromised. In some embodiments, the subject can be undergoing chemotherapy.

Examples of agents that have been used to treat HBV and/or HDV infection include interferons (such as IFN-α and pegylated interferons that include PEG-IFN-α-2a), and nucleosides/nucleotides (such as lamivudine, telbivudine, adefovir dipivoxil, clevudine, entecavir, tenofovir alafenamide, and tenofovir disoproxil). However, some of the drawbacks associated with interferon treatment are the adverse side effects, the need for subcutaneous administration and high cost. A drawback with nucleoside/nucleotide treatment can be the development of resistance.

Resistance can be a cause for treatment failure. The term “resistance” as used herein refers to a viral strain displaying a delayed, lessened and/or null response to an anti-viral agent. In some embodiments, a modified oligonucleotide or complex thereof as described herein can be provided to a subject infected with an HBV and/or HDV strain that is resistant to one or more anti-HBV and/or anti-HDV agents. Examples of anti-viral agents wherein resistance can develop include lamivudine, telbivudine, adefovir dipivoxil, clevudine, entecavir, tenofovir alafenamide, and tenofovir disoproxil. In some embodiments, development of resistant HBV and/or HDV strains is delayed when a subject is treated with a modified oligonucleotide as described herein compared to the development of HBV and/or HDV strains resistant to other HBV and/or HDV anti-viral agents, such as those described.

### Combination Therapies

In some embodiments, a modified oligonucleotide or complex thereof as described herein can be used in combination with one or more additional agent(s) for treating and/or inhibiting replication HBV and/or HDV. Additional agents include, but are not limited to, an interferon, nucleoside/nucleotide analogs, a capsid assembly modulator, a sequence specific oligonucleotide (such as anti-sense oligonucleotide and/or siRNA), an entry inhibitor and/or a small molecule immunomodulator. For example, in some embodiments, a modified oligonucleotide or complex thereof as described herein can be used as a first treatment in combination with one or more second treatment(s) for HBV, wherein the second treatment includes a second oligonucleotide having sequence independent antiviral activity against hepatitis B, an siRNA oligonucleotide (or nucleotides), an anti-sense oligonucleotide, a nucleoside, an interferon, a viral entry inhibitor, an immunomodulator, a capsid assembly modulator, an anti-HBsAg mAb, or a combination thereof. Nonlimiting examples of additional agents include an HBV capsid assembly modulator, ALG-000184, ALG-125755, GSK-836, VIR-2218, recombinant interferon alpha 2b, IFN-gamma, PEG-IFN-alpha-2a, lamivudine, telbivudine, adefovir dipivoxil, clevudine, entecavir, tenofovir alafenamide, tenofovir disoproxil, RG6004, GSK3228836, VIR-3434, BJT-778, Bulevirtide, DCR-HBVS, GLS4, NZ-4, and RG7907. In some embodiments, the additional agent is a capsid assembly modulator (CAM). In some embodiments, the additional agent is an anti-sense oligonucleotide (ASO).

In some embodiments, a modified oligonucleotide or complex thereof as described herein can be administered with one or more additional agent(s) together in a single pharmaceutical composition. In some embodiments, a modified oligonucleotide or complex thereof as described herein can be administered with one or more additional agent(s) as two or more separate pharmaceutical compositions. Further, the order of administration of a modified oligonucleotide or complex thereof as described herein with one or more additional agent(s) can vary.

### EXAMPLES

Additional embodiments are disclosed in further detail in the following examples, which are not in any way intended to limit the scope of the claims.

### Example 1

A series of modified oligonucleotides containing phosphorothioated sequences of alternating A and C units were synthesized on an ABI 394 synthesizer using standard phosphoramidite chemistry. The solid support was controlled pore glass (CPG, 1000A, Glen Research, Sterling VA) and the building block monomers are described in Tables 3 and 4. The reagent (dimethylamino-methylidene) amino)-3H-1,2,4-dithiazaoline-3-thione (DDTT) was used as the sulfur-transfer agent for the synthesis of oligoribonucleotide phosphorothioates (PS linkages). An extended coupling of 0.1 M solution of phosphoramidite in CH₃CN in the presence of 5-(ethylthio)-1H-tetrazole activator to a solid bound oligonucleotide followed by standard capping, oxidation and deprotection afforded modified oligonucleotides. The stepwise coupling efficiency of all modified phosphoramidites was more than 95%. Several modified oligonucleotides containing sequences of alternating A and C units but having phosphodiester (PO) linkages instead of phosphorothioate (PS) linkages were also made.

### Deprotection

After completion of synthesis the controlled pore glass (CPG) was transferred to a screw cap vial or screw caps RNase free microfuge tube. The oligonucleotide was cleaved from the support with simultaneous deprotection of base and phosphate groups with 1.0 mL of a mixture of ethanolic ammonia (ammonia:ethanol (3:1) for 5-15 hr at 55° C. The vial was cooled briefly on ice and then the ethanolic ammonia mixture was transferred to a new microfuge tube. The CPG was washed with 2× 0.1 mL portions of deionized water, put in dry ice for 10 min then dried in speed vac.

Oligonucleotides containing ribo-phosphoramidites required a two-step deprotection process to remove the 2′ protecting group. After completion of synthesis, the CPG was transferred to an RNase free microfuge falcon tube. The oligonucleotide was cleaved from the support and simultaneous deprotection of base and phosphate groups with 0.5 ml of concentrated ammonium hydroxide solution for 2-4 hr at 80° C. The vial was cooled briefly, and the oligonucleotide product was treated with 1.5 ml dimethyl sulfoxide and 0.75 ml triethylamine trihydrofluoride for 2-4 hr at 65° C. The CPG was washed with 2×0.1 mL portions of deionized water, put in dry ice for 10 min then dried in speed vac.

### Quantitation of Crude Oligomer or Raw Analysis

Samples were dissolved in deionized water (1.0 mL) and quantitated as follows: Blanking was first performed with water alone (1 mL). 20 μl of sample and 980 μL of water were mixed well in a microfuge tube, transferred to cuvette and absorbance reading obtained at 260 nm. The crude material was dried down and stored at −20° C.

### HPLC Purification of Oligomer

The crude oligomers were analyzed and purified by HPLC (Dionex PA 100). The buffer system is A=Water B=0.25 M Tris-HCl pH 8, C: 0.375 M Sodium per chlorate, flow 5.0 mL/min, wavelength 260 nm. A small amount of material was injected (˜5 OD) and analyzed by LC-MS. Once the identity of this material was confirmed, the crude oligomer was purified using a larger amount of material, e.g., 60 ODs per run, flow rate of 5 mL/min. Fractions containing the full-length oligonucleotides were then pooled together, evaporated, and finally desalted as described below.

### Desalting of Purified Oligomer

The purified dry oligomer was desalted using Sephadex G-25M (Amersham Biosciences). The cartridge was conditioned with 10 mL of water. The purified oligomer dissolved thoroughly in 2.5 mL RNase free water was applied to the cartridge with slow dropwise elution. The salt free oligomer was eluted with 3.5 ml water directly into a screw cap vial.

### HPLC Analysis and Electrospray LC/MS

Approximately 0.2 OD oligomer was first dried down, redissolved in water (50 μl), and then pipetted in special vials for HPLC and LC-MS analysis. Table 5 provides mass spec values for oligonucleotides that were made by these methods:

TABLE 5

| SEQ ID NO: | Theoretical mass | Experimental mass |
|---|---|---|
| 3 | 14053.0 | 14053.1 |
| 4 | 13985.7 | 13987.2 |
| 5 | 13989.7 | 13991.2 |
| 6 | 13993.7 | 13995.4 |
| 7 | 13997.7 | 13999.7 |
| 8 | 14001.7 | 14003.0 |
| 9 | 14005.7 | 14006.8 |
| 10 | 14009.8 | 14010.7 |
| 11 | 14013.8 | 14015.3 |
| 12 | 14017.8 | 14019.5 |
| 13 | 14021.8 | 14022.9 |
| 14 | 14021.7 | 14023.1 |
| 15 | 14007.7 | 14009.6 |
| 16 | 14011.8 | 14013.4 |
| 17 | 14015.8 | 14017.2 |
| 18 | 14019.8 | 14021.4 |
| 19 | 14009.8 | 14010.8 |
| 20 | 14013.8 | 14014.9 |
| 21 | 14017.8 | 14019.0 |
| 22 | 14015.8 | 14016.9 |
| 23 | 14015.8 | 14016.7 |
| 24 | 14017.8 | 14018.4 |
| 25 | 14017.8 | 14018.4 |
| 26 | 14019.9 | 14020.6 |
| 27 | 14019.9 | 14020.6 |
| 28 | 14015.8 | 14016.9 |
| 29 | 14019.9 | 14021.1 |
| 30 | 14019.9 | 14020.7 |
| 31 | 14017.8 | 14019.7 |
| 32 | 14017.8 | 14020.4 |
| 33 | 14019.9 | 14022.7 |
| 34 | 14017.8 | 14018.8 |
| 35 | 14019.9 | 14020.3 |
| 36 | 14033.8 | 14034.2 |
| 37 | 14039.9 | 14040.7 |
| 38 | 14043.9 | 14044.4 |
| 39 | 14047.9 | 14048.6 |
| 40 | 14043.9 | 14044.6 |
| 41 | 14045.9 | 14046.5 |
| 42 | 14047.9 | 14049.3 |
| 43 | 14085.9 | 14086.7 |
| 44 | 13983.7 | 13980.2 |
| 45 | 14013.6 | 14013.7 |
| 46 | 14093.9 | 14094.0 |

### Example 2

### HBsAg Secretion Assay and Cytotoxicty Assay Protocol

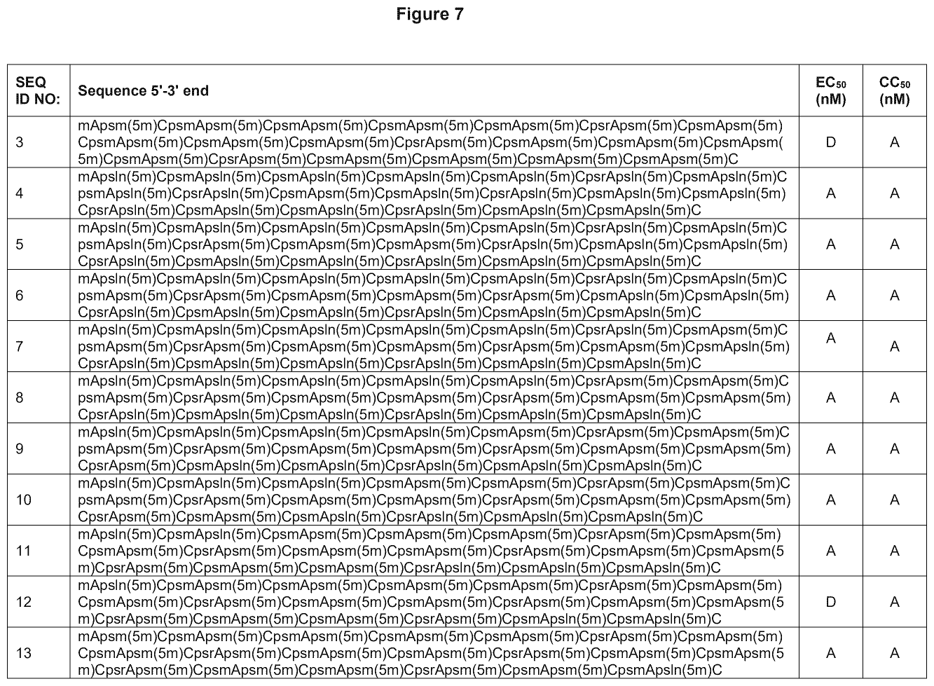

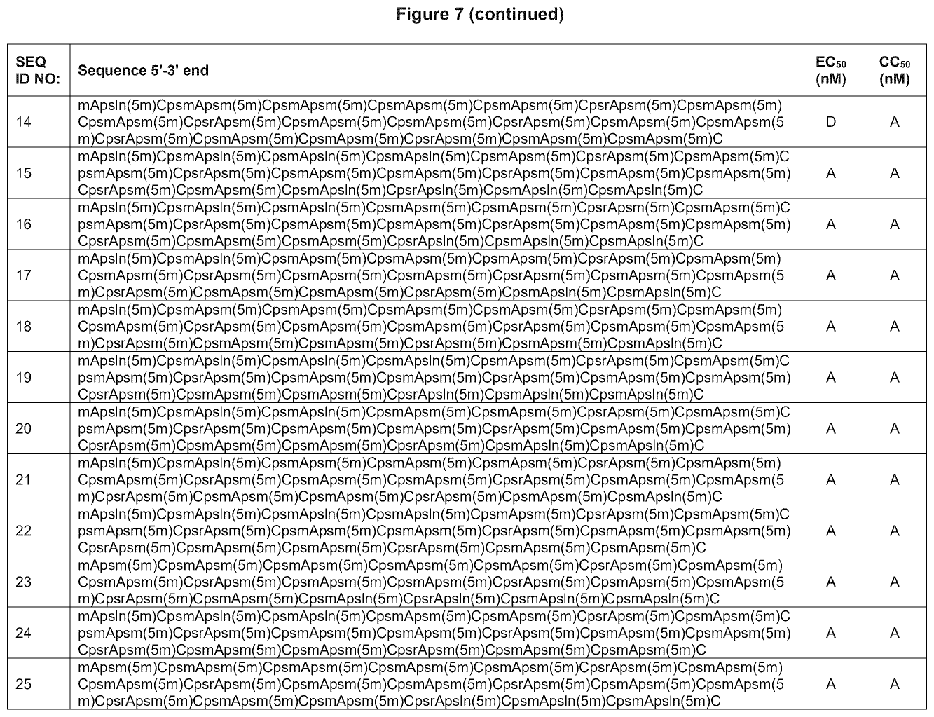

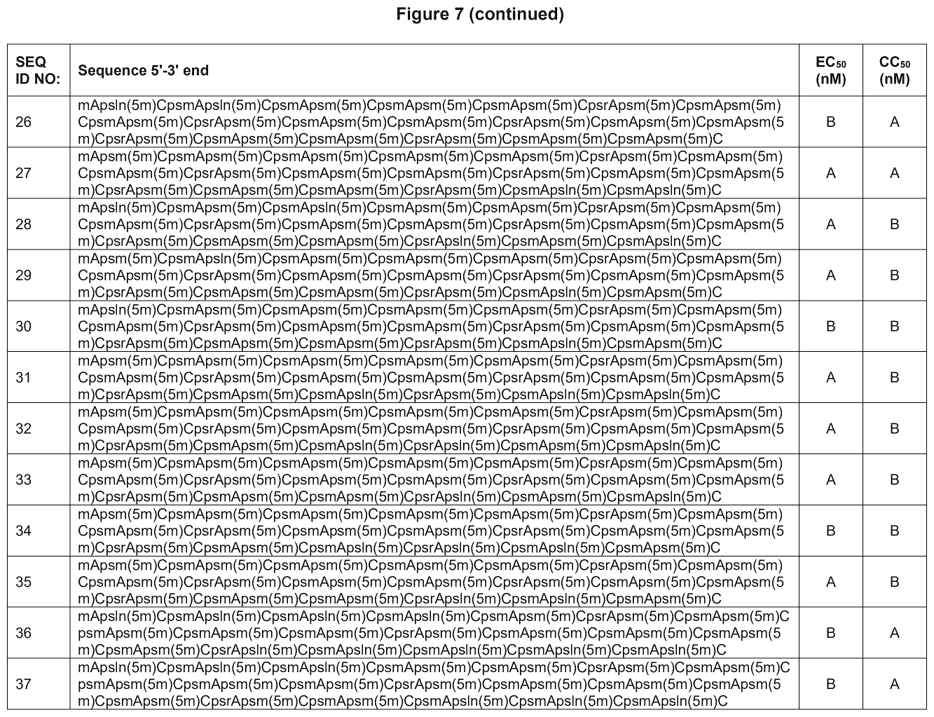

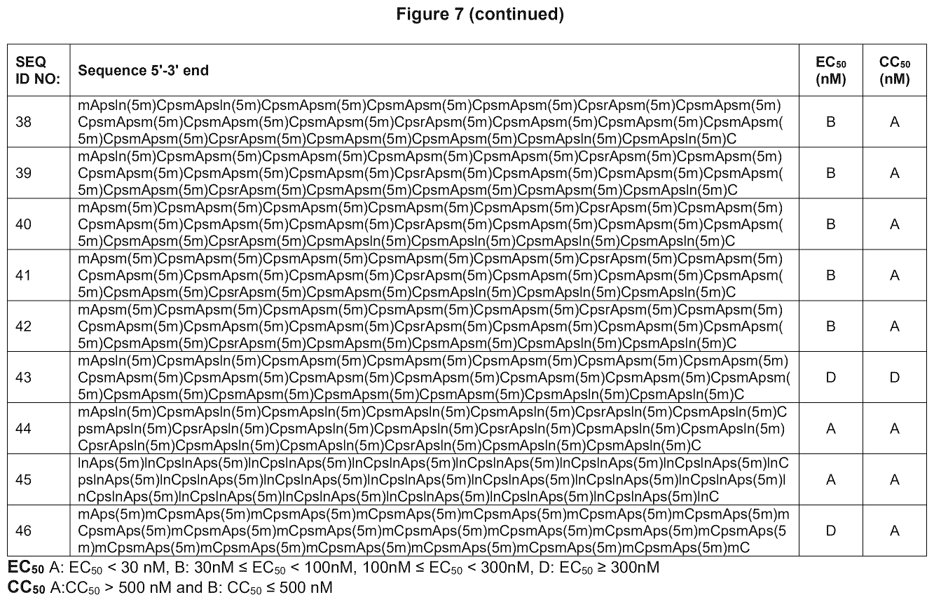

The methodology for the sequence independent antiviral activity against HBV (as determined by HBsAg Secretion Assay) and the cytotoxicity of a number of exemplified modified oligonucleotide compounds was determined as described below using the following experimental protocol and summarized in FIG. 7.

### HBsAg Release Assay Protocol

### Cell Culture

HepG2.2.15 cells were maintained in DMEM medium with 10% fetal bovine serum (FBS) and 1% penicillin/streptomycin, 1% Glutamine, 1% non-essential amino acids, 1% Sodium Pyruvate and 380 μg/mL G418. Cells were maintained at 37° C. in a 5% CO₂ atmosphere.

### HBsAg Secretion Assay

HepG2.2.15 cells were grown in DMEM medium as described above. Cells were plated at a concentration of 45,000 cells/well in collagen-I coated 96 well plates. Immediately after addition of the cells, test compounds were added.

Selected compounds were tested following Lipofectamine® RNAiMAX transfection. Lipofectamine® RNAiMAX Transfection Reagent (Thermo Fisher) was used following the manufacturer's instructions.

The 50% inhibitory concentration (EC₅₀) and 50% cytotoxic concentration (CC₅₀; below) were assessed by solubilizing in 1×PBS to 100× the desired final testing concentration. Each compound was then serially diluted (1:3) up to 8 distinct concentrations to 10× the desired final testing concentration in DMEM medium with 10% FBS. A 10 μL sample of the 10× compounds in cell culture media was used to treat the HepG2.2.15 cells in a 96-well format. Cells were initially incubated with compounds for 3 days at 37° C. in a 5% CO₂ atmosphere.

Three days post compound addition/transfection, the media was replaced with fresh media/compound with RNAiMax and incubated for a further 3 days for a total incubation time of 6 days. Both the cellular supernatant and cell lysate were collected (see below) for quantification of HBsAg.

Secreted HBsAg was measured quantitatively using HBsAg ELISA kit (Autobio-CL0310).

The EC₅₀, the concentration of the drug required for reducing HBsAg secretion by 50% in relation to the untreated cell control value was calculated from the plot of the percentage reduction of the HBsAg level against the drug concentrations using Microsoft Excel (forecast function).

A parallel set of plates that was set up for testing compound induced cellular cytotoxicity (see below).

### Cytotoxicity Assay

HepG2.2.15 cells were cultured and treated as above. At Day 6, cellular cytotoxicity was assessed using a cell proliferation assay (CellTiter-Glo Luminescent Cell Viability Assay; Promega) according to the manufacturer's instructions or a suitable alternative.

The CC₅₀, the concentration of the drug required for reducing cell viability by 50% in relation to the untreated cell control value, was calculated from the plot of the percentage reduction of viable cells against the drug concentrations using Microsoft Excel (forecast function).

### Example 3

### Live Infection Assay Protocol

HepG2-NTCP cells were maintained in DMEM/F12 medium with 10% fetal bovine serum (FBS) and 1% penicillin/streptomycin, 1% Glutamine, 1% non-essential amino acids, and 1% Sodium Pyruvate. Cells were maintained at 37° C. in a 5% CO₂ atmosphere.

HepG2-NTCP cells were resuspended with above mentioned medium and plated at a concentration of 15,000 cells/well in collagen-I coated 96 well plates. On the second day (day 0), the cells were infected with HBV (purified HBV from HepAD38 cells) at 200 MOI (multiplicity of infection, genome equivalents per cell) in the presence of 4% PEG8000 and 2% DMSO and incubated at 37° C. overnight. The inoculum was vacuumed, and cells were washed three times with DMEM/F12 with 2% FBS before replacing with the HepG2-NTCP culture medium.

The cells were treated on day 5. On day 5, the test compounds were diluted 3-fold with Opti-MEM I media and mixed with Lipofectamine® RNAiMAX transfection reagent following the manufacturer's instructions. After media replacement on Day 8, the test compounds were transfected as described. After incubation for an additional 3 days, the supernatant was harvested and HBsAg was measured by ELISA (DiaSino). The cell viability was measured with CellTiter-Glo (Promega).

The EC₅₀, the concentration of the drug required for reducing HBsAg secretion by 50% in relation to the untreated cell control value, was calculated from the plot of the percent reduction of the HBsAg level against the drug concentrations using the Microsoft Excel forecast function or GraphPad Prism.

### Example 4

### Helicity and In Vitro Activity of New Stops™

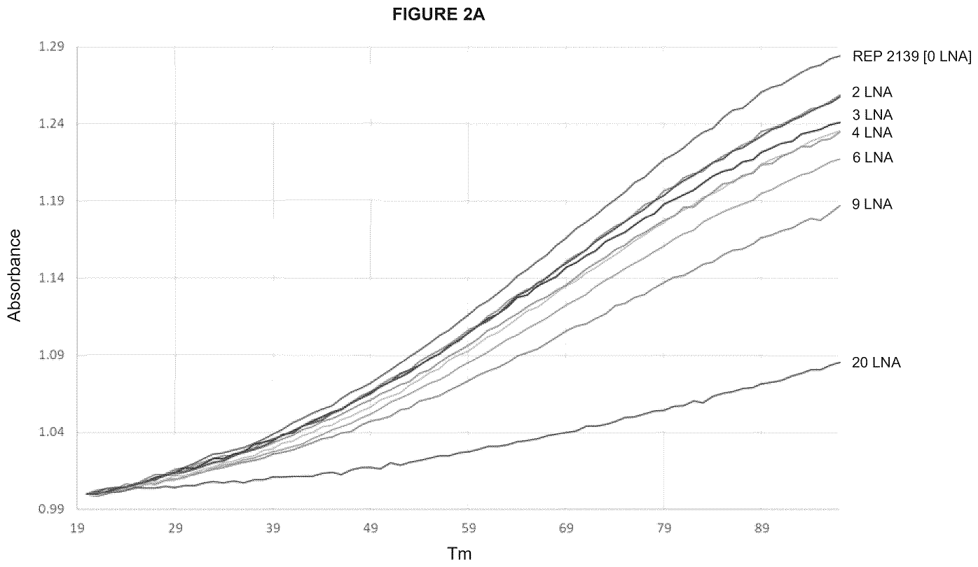

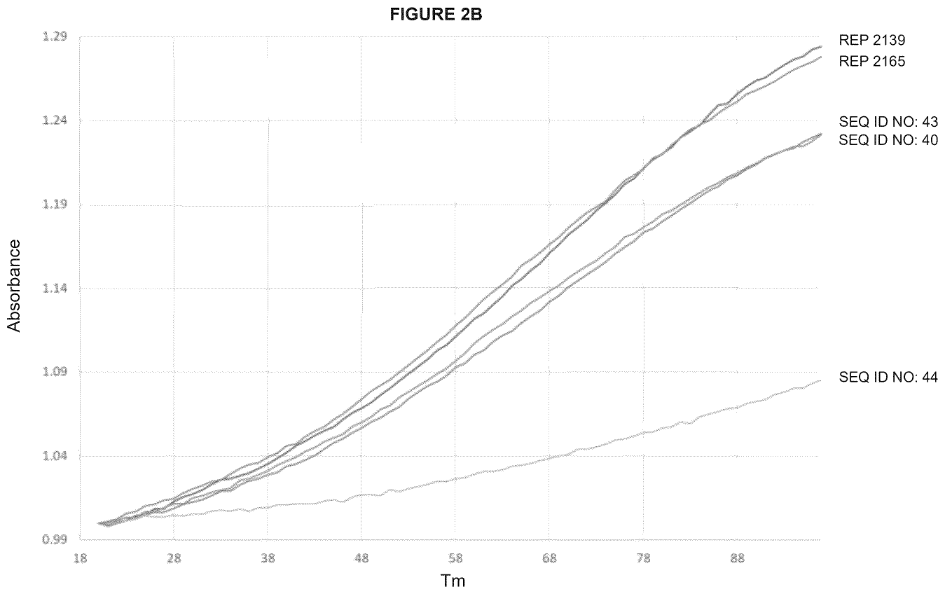

A diverse set of oligonucleotide constructs were synthesized and screened using a Tm assay, as well as a HepG2.2.15 HBsAg Release assay. Twenty-four of these oligonucleotide compounds were sequenced with alternating patterns of LNA. Helicity was determined using melting temperature (Tm) experiment for each set of newly synthesized oligonucleotides. Melting temperature experiments were performed using Shimadzu UV-2600 spectrometer using 2 μM solution of oligonucleotide in PBS (1×). The sequences shown in FIG. 2A are REP-2139 (SEQ ID NO: 46), 2 LNA (SEQ ID NOs: 18 and 27), 3 LNA (SEQ ID NO: 25), 4 LNA (SEQ ID NOs: 17 and 23), 6 LNA (SEQ ID NO: 16), 9 LNA (SEQ ID NO: 9), and 20 LNA (SEQ ID NO: 44) wherein the STOPS™ compounds with 20 LNA were the most rigid, and the STOPS compounds with 1 LNA were the most flexible. Generally, as the number of LNA in a sequence decreases, the oligonucleotide's flexibility increases (FIGS. 2A and 2B). The following Table 6 provides the in vitro activity of these compounds, which shows compounds with reduced LNA, e.g. without LNA in the central region, maintain good potency while having preferable helicity (FIG. 2A).

TABLE 6

| SEQ ID NO: | Design | EC₅₀ (nM) | CC₅₀ (nM) |
|---|---|---|---|
| 4 | 19 LNA-5mC | A | A |
| 5 | 17 LNA-5mC | A | A |
| 6 | 15 LNA-5mC | A | A |
| 7 | 13 LNA-5mC | A | A |
| 8 | 11 LNA-5mC | A | A |
| 9 | 9-LNA-5mC | A | A |
| 10 | 7-LNA-5mC | A | A |
| 11 | 5-LNA-5mC | A | A |
| 12 | 3-LNA-5mC | D | A |
| 13 | 1-LNA-5mC (3′end) | A | A |
| 14 | 1-LNA-5mC (5′ end) | D | A |
| 15 | 8-LNA-5mC 4 and 4 gapmer | A | A |
| 16 | 6-LNA-5mC 3 and 3 gapmer | A | A |
| 17 | 4-LNA-5mC 2 and 2 gapmer | A | A |
| 18 | 2-LNA-5mC 1 and 1 gapmer | A | A |
| 19 | 7-LNA-5mC 4 and 3 gapmer | A | A |
| 20 | 5-LNA-5mC 3 and 2 gapmer | A | A |
| 21 | 3-LNA-5mC 2 and 1 gapmer | A | A |
| 22 | 4-LNA-5mC 4 LNA at 5′-end | A | A |
| 23 | 4-LNA-5mC 4 LNA at 3′-end | A | A |
| 24 | 3-LNA-5mC 3 LNA at 5′-end | A | A |
| 25 | 3-LNA-5mC 3 LNA at 3′-end | A | A |
| 26 | 2-LNA-5mC 2 LNA at 5′-end | B | A |
| 27 | 2-LNA-5mC 2 LNA at 3′-end | A | A |

* A: EC₅₀ < 30 nM, B: 30 nM ≤ EC₅₀ < 100 nM, C: 100 nM ≤ EC₅₀ < 300 nM, D: EC₅₀ ≥ 300 nM; A: CC₅₀ > 500 nM and B: CC₅₀ ≤ 500 nM

### Example 5

### Duck Hepatits B Virus Model

Nucleic acid polymers (NAPs) are an attractive treatment modality for chronic hepatitis B (CHB), with REP-2139 and REP2165 having shown efficacy in CHB patients. A subset of patients achieved functional cure, whereas the others exhibit a moderate response or are non-responders. NAP efficacy has been difficult to recapitulate in animal models, with the duck hepatitis B virus (DHBV) model showing some promise but remaining underexplored for NAP efficacy testing. To this end, Pekin ducks (Anas platyrhynchos domesticus) were intravenously infected with DHBV-containing serum shortly after hatching. After establishment of infection, animals were treated with entecavir, REP2139 (SEQ ID NO: 46), and/or REP2165 (SEQ ID NO: 3) and serum DHBV DNA and DHBV surface antigen (DHBsAg) levels were determined weekly. Animals were followed for several weeks after end of treatment. NAP serum and tissue concentrations were determined by mass spectrometry. The optimized protocol resulted in low drop-out rates and robust read-outs. REP2139 was efficacious in reducing DHBV DNA and DHBsAg levels in approximately half of the treated ducks, whether administered intraperitoneally or subcutaneously. Intrahepatic or serum NAP concentrations did not correlate with efficacy, nor did the appearance of anti-DHBsAg antibodies. Furthermore, NAP efficacy was only observed in experimentally infected ducks, not in endogenously infected ducks (vertical transmission). REP2139 add-on to entecavir treatment induced a deeper and more sustained virological response compared to entecavir monotherapy. Destabilized REP2165 showed a different activity profile with a more homogenous antiviral response followed by a faster rebound. In conclusion, subcutaneous administration of NAPs in the DHBV duck model provided a useful tool for in vivo evaluation of NAPs (Debing Yannick et al., Antiviral Research 224 (2024) 105835). The model recapitulates many aspects of this class of compound's efficacy in CHB patients, most notably the clear division between responders and non-responders.

### Reference Compounds

Entecavir hydrate was purchased from Carbosynth (Compton, UK, NE10532). REP-2139 and REP-2165 were synthesized as per literature protocol.

### In Vivo Studies

All animal studies were conducted at TRANSfarm (Leuven, Belgium). All animal procedures followed local animal welfare legislation, ARRIVE guidelines, and were approved by the local ethical committee (project reference P069/2019). In each study, fertilized Pekin duck (Anas platyrhynchos) eggs were obtained from local commercial poultry farms. The eggs were incubated for the remainder of the 28 days under ideal conditions (for example, at a temperature of 36.8° C., relative humidity 57% and wet bulb temperature 29.5° C.). The incubator was opened only for refilling water and turning of the eggs. Within 1 to 3 days after hatching, ducklings were weighed, sampled for blood, and inoculated by intravenous injection with 200 μL of DHBV-positive serum (corresponding to 10⁷ viral genome equivalents/duckling). Ducklings were housed in a single cage with heat lamps, within a controlled environment keeping the temperature at 23° C.±7° C. with light cycles of 11 hours of light (8:00 to 19:00). Water and pelleted food for waterfowl was provided ad libitum. To assess the evolution of serum DHBV DNA and DHBsAg titers, ducks were sampled 28 days post infection. Based on these criteria that define the level of infection and on body weight, ducks were randomly allocated into multiple treatment groups. Vertically/endogenously infected ducklings were excluded from the study. Entecavir was administered orally every day as a 1 mL solution (fixed dose of 1 mg/day). NAPs were administered intraperitoneally or subcutaneously every other day with dosing of 10 mg/kg at a volume of 0.5 mL/kg in PBS. Blood was sampled weekly and collected in CAT serum clot activator tubes via the medial metatarsal vein from the start of the treatment until the end of the experiment. After blood collection, the CAT serum clot activator blood test tubes were kept at 4° C. overnight to allow serum separation. Subsequently, the serum was collected by centrifugation (2000 rpm for 15 minutes at room temperature), transferred to new 1.8 mL cryo-tubes, inventoried and either stored at −80° C. or analyzed directly. After 4 weeks of treatment, 3 animals per group were sacrificed by euthanizing them with an electric stun followed by decapitation or cervical dislocation, and their livers and kidneys harvested, then flash frozen in liquid nitrogen. The remainder of the animals were followed up for an additional 4 to 8 weeks after end of treatment. On the last day of the experiment, the remaining ducks were euthanized as described previously, with their livers and kidneys harvested and flash frozen in liquid nitrogen.

### DHBV DNA Extraction and Quantification

Serum samples from all ducks were thawed at room temperature and centrifuged at 11,000 g for 1 minute at room temperature. The clarified serum samples were diluted 1:2 in PBS and DHBV DNA was extracted using the Macherey-Nagel Virus 96 kit (740452.4, Macherey-Nagel) according to the manufacturer's instructions. DNA was eluted with 75 μL of prewarmed elution buffer. The DHBV DNA samples were quantified by SYBR green qPCR using a 1:10 dilution series of control DHBV gBlock (IDT). Per reaction, 4 μL of sample DNA was added to 1× BioRad Universal SYBRgreen Supermix, 0.4 μM of both forward and reverse primer (5′-CTGACGGACAACGGGTCTAC-3′ (SEQ ID NO: 1) and 5′-GGGTGGCAGAGGAGGAAGT-3′ (SEQ ID NO: 2), respectively). MilliQ water was added to a total volume of 20 μL per reaction. After a pre-incubation of 3 minutes at 95° C., the samples went through 40 cycles of 10 seconds denaturation at 95° C. followed by 30 seconds elongation at 60° C., on a BioRad CFX qPCR machine.

### DHBsAg Quantification-ELISA

Aliquots of serum samples from all ducks, prepared as described in the previous section, were diluted 1:50 in PBS containing 8% chicken serum. A 96-well immunoplate (456537, ThermoFisher) was coated with 150 μL of the diluted serum samples. In a separate column, a 1:2 dilution series of purified large DHBsAg (Bioclone, San Diego, CA) was added to serve as a standard curve (20 to 0.002 μg/mL). As negative control, PBS with only chicken serum was included. Afterwards, the plate was incubated overnight at 37° C. Samples were removed and the plate was blocked with 150 μL of PBS containing 2% bovine serum albumin (BSA) and 1% goat serum for 1.5 hours at room temperature. The plate was washed three times with 150 μL of PBST (PBS with 0.1% Tween-20). Then, 100 μL of primary rabbit polyclonal anti-DHBsAg antibody (Creative Diagnostics) 1:2000 diluted in blocking buffer was added for 1 hour at room temperature. The plate was washed again three times with 150 μL of PBST. Next, 100 μL of secondary antibody goat anti-Rabbit IgG HRP (G21234, Invitrogen) diluted 1:3000 in blocking buffer was added for 30 minutes at room temperature. The plate was washed 5 times with 150 μL of PBST. The plate was incubated with 100 μL of TMB developing substrate (N301, ThermoFisher) for approximately 5 minutes until the positive samples showed a clear blue signal. At last, 100 μL of 0.18 M H₂SO₄ (diluted from 95% in H₂O) was added to each well to stop the reaction. The absorbance was measured at 450 nm with a plate reader.

### Example 6

### DHBV Duck Studies

### REP-2139 vs SEQ ID NO: 44 vs REP-2165

Five groups of 12 ducks each were given 28 days of treatment with either: (1) no treatment, (2) entecavir orally at 1 mg/day, (3) REP-2139 subcutaneously (SC) at 10 mpk QOD, (4) an oligonucleotide of SEQ ID NO: 44 SC at 10 mpk QOD, (5) REP-2165 SC at 10 mpk QOD.

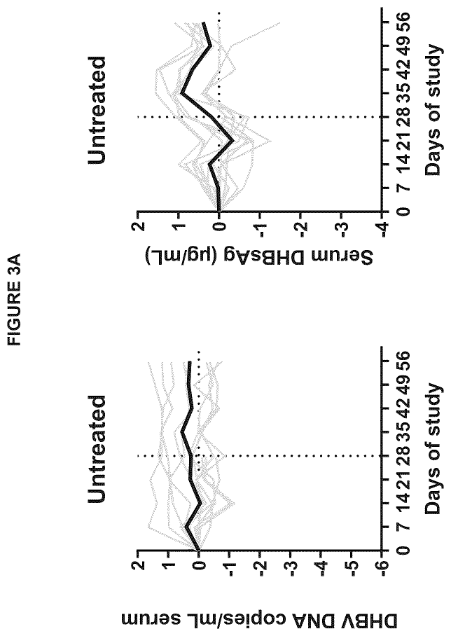

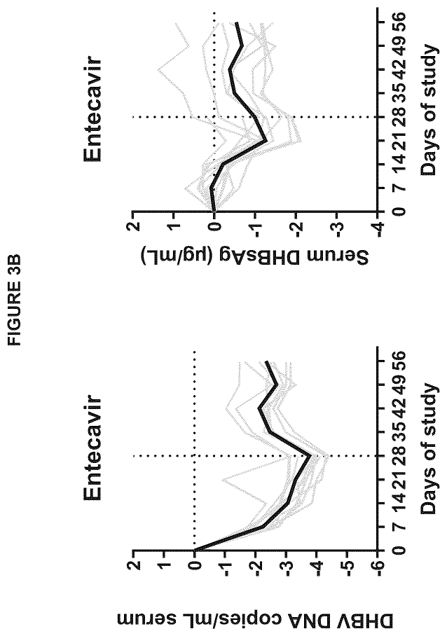

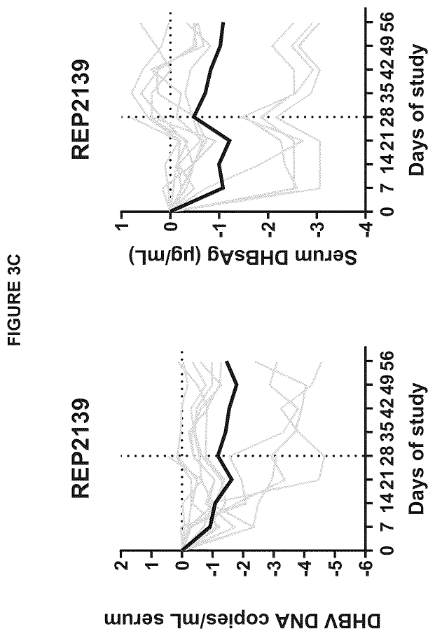

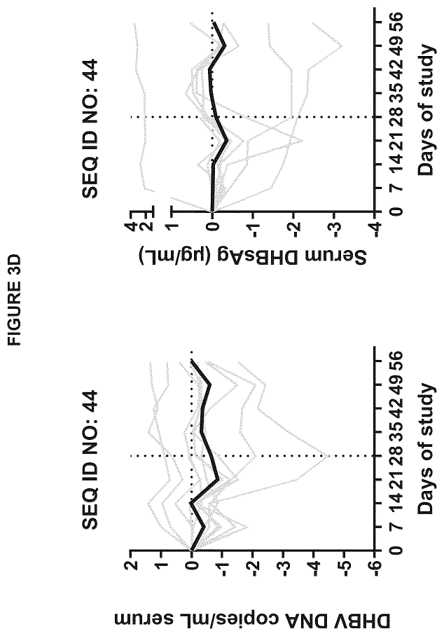

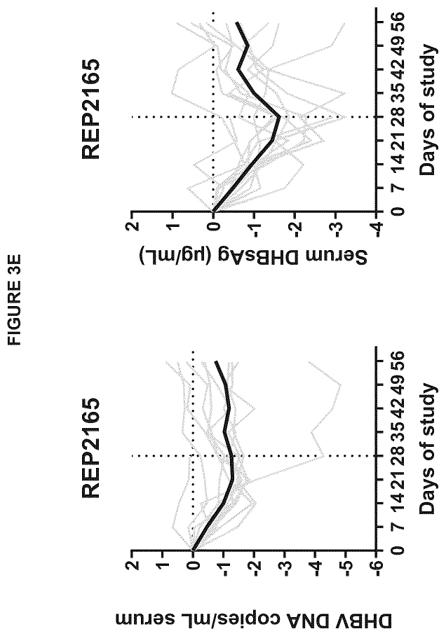

At the end of the 28 days, 3 ducks in each group were sacrificed for pharmacokinetic (PK) evaluation. Then, after the 1-month follow up was completed, all ducks were sacrificed for PK evaluation. The reduction of DHBV DNA and DHBsAg is as shown in FIGS. 3A-3E, and in Table 7.

TABLE 7

| | | Ducks with | Ducks with |
|---|---|---|---|
| | | DHBV DNA >2 | DHBsAg >2 |
| | | log₁₀ decline | log₁₀ decline |
| | | from baseline | from baseline |
| Untreated | End of Treatment (Day 28) | 0/12 | 0/12 |
| | End of Follow Up (Day 56) | 0/9 | 0/9 |
| Entecavir | End of Treatment | 11/11 | 0/11 |
| | End of Follow Up | 6/8 | 0/8 |
| REP2139 (SEQ | End of Treatment | 3/12 | 1/12 |
| ID NO: 46) | End of Follow Up | 3/9 | 3/9 |
| SEQ ID NO: 44 | End of Treatment | 2/10 | 1/10 |
| | End of Follow Up | 0/7 | 1/7 |
| REP2165 (SEQ | End of Treatment | 1/12 | 4/12 |
| ID NO: 3) | End of Follow Up | 1/9 | 1/9 |

The untreated group demonstrated stable DHBV DNA trends over time (FIG. 3A, left panel). DHBsAg had more variability, but stabilized over time (FIG. 3A, right panel). Entecavir treatment, meanwhile, resulted in a pronounced reduction to both DHBV DNA and DHBsAg, followed by a rebound (FIG. 3B). In the group treated with REP-2139, there was a clear differentiation between responders (1/3) versus nonresponders (2/3) to treatment (FIG. 3C). Altogether, there was approximately a 33% response to REP2139. Treatment with the compound of SEQ ID NO: 44 had an inferior response (14%) compared to REP-2139, although not completely absent (FIG. 3D). In ducks given REP-2165, there was a homogenous response (11%) as monitored through both DHBV DNA and DHBsAg, with a clear and mostly uniform rebound, and one true responder (FIG. 3E).

### REP2139 vs SEQ ID NO: 38 vs SEQ ID NO: 43

Four groups of ducks were given 28 days of treatment with either: (1) no treatment, (2) REP2139 (SEQ ID NO: 46) subcutaneously at 10 mpk QOD, (3) SEQ ID NO: 38 subcutaneously at 10 mpk QOD, or (4) SEQ ID NO: 43 subcutaneously at 10 mpk QOD. The structures of compounds with sequences of SEQ ID NOs: 38 and 43 are as shown in FIG. 4, and in Table 8.

TABLE 8

| SEQ ID NO: | Sequence 5′→3′ |
|---|---|
| 38 | 5′mApsln(5m)CpsmApsln(5m)CpsmApsm(5m)CpsmApsm(5m)CpsmAp |
| | sm(5m)CpsrApsm(5m)CpsmApsm(5m)CpsmApsm(5m)CpsmApsm(5m) |
| | CpsmApsm(5m)CpsrApsm(5m)CpsmApsm(5m)CpsmApsm(5m)CpsmAp |
| | sm(5m)CpsmApsm(5m)CpsrApsm(5m)CpsmApsm(5m)CpsmApsm(5m) |
| | CpsmApsln(5m)CpsmApsln(5m)C-3′ |
| 43 | 5′mApsln(5m)CpsmApsln(5m)CpsmApsm(5m)CpsmApsm(5m)CpsmAp |
| | sm(5m)CpsmApsm(5m)CpsmApsm(5m)CpsmApsm(5m)CpsmApsm(5m) |
| | CpsmApsm(5m)CpsmApsm(5m)CpsmApsm(5m)CpsmApsm(5m)CpsmA |
| | psm(5m)CpsmApsm(5m)CpsmApsm(5m)CpsmApsm(5m)CpsmApsm(5m) |
| | CpsmApsln(5m)CpsmApsln(5m)C-3′ |

Compounds of SEQ ID NO: 38 and 43 have improved helicity (change in (improvement in “S” shape of Tm curve) relative to the compound of SEQ ID NO: 44 given their reduced LNA units compared to SEQ ID NO: 44 (FIG. 2B). Both compounds have LNA units added at both ends. SEQ ID NO: 43 has a similar design as SEQ ID NO: 38 (4 LNA), wherein SEQ ID NO: 38 also features three ribo-A units in the central region of the oligonucleotide sequence that are not present in SEQ ID NO: 43.

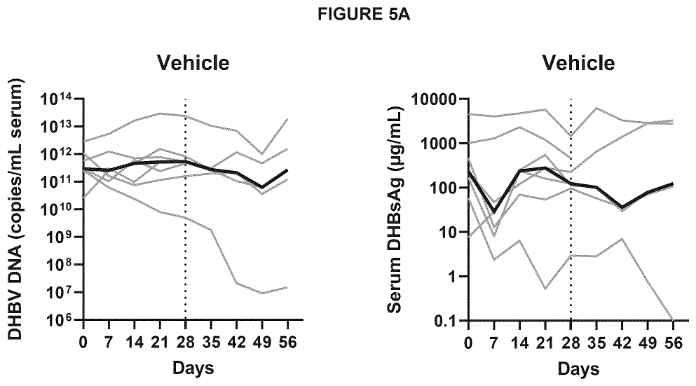

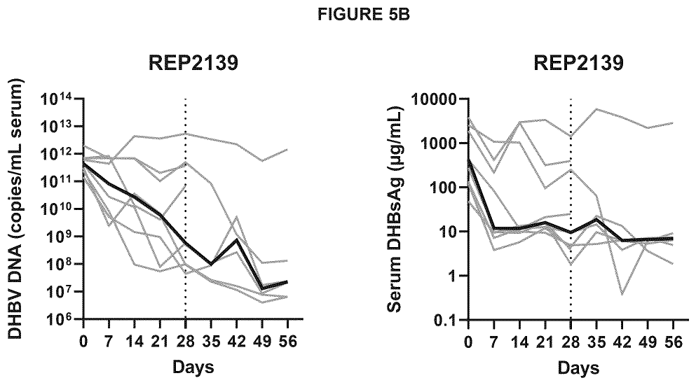

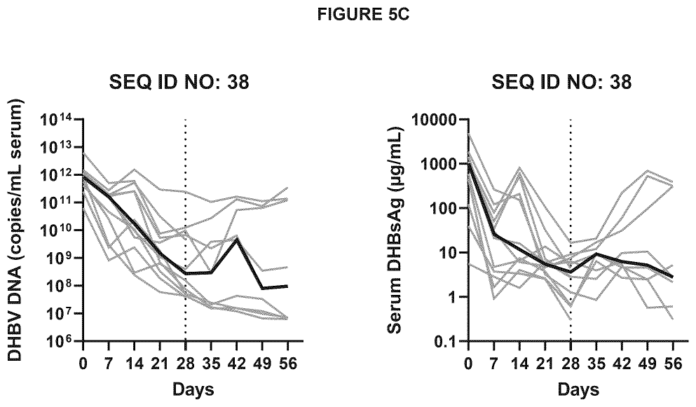

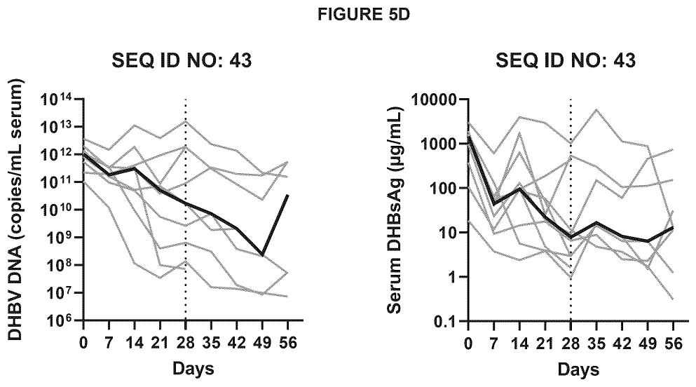

At the end of the 28 days, 3 ducks in each group were sacrificed for pharmacokinetic (PK) evaluation. Then, after the 1-month follow up was completed, all remaining ducks were sacrificed for PK evaluation. The reduction of DHBV DNA and DHBsAg is as shown in FIGS. 5A-5D, and in Table 9.

TABLE 9

| | | Ducks with | Ducks with |
|---|---|---|---|
| | | DHBV DNA >1 | DHBsAg >1 |
| | | log₁₀ decline | log₁₀ decline |
| | | from baseline | from baseline |
| Untreated | End of Treatment (Day 28) | 1/7 | 1/7 |
| | End of Follow Up (Day 56) | 1/5 | 1/5 |
| REP-2139 | End of Treatment | 6/9 | 7/9 |
| | End of Follow Up | 5/6 | 4/6 |
| SEQ ID NO: 38 | End of Treatment | 11/12 | 11/12 |
| | End of Follow Up | 8/9 | 7/9 |
| SEQ ID NO: 43 | End of Treatment | 6/10 | 7/10 |
| | End of Follow Up | 5/7 | 5/7 |

The untreated group demonstrated stable DHBV DNA trends over time (FIG. 5A, left panel). DHBsAg had more variability, but stabilized over time (FIG. 5A, right panel). The control group also had one spontaneous clearance of infection, which has never been observed before without treatment. In the group treated with REP-2139, there was a clear differentiation between responder versus nonresponders to treatment (FIG. 5B). In ducks given SEQ ID NO: 38, there was a homogenous response, followed by a separation of responders and non-responders (FIG. 5C). Ducks given SEQ ID NO: 43 had a similar response to ducks given REP-2139, and there was an early division between responder and non-responders (FIG. 5D).

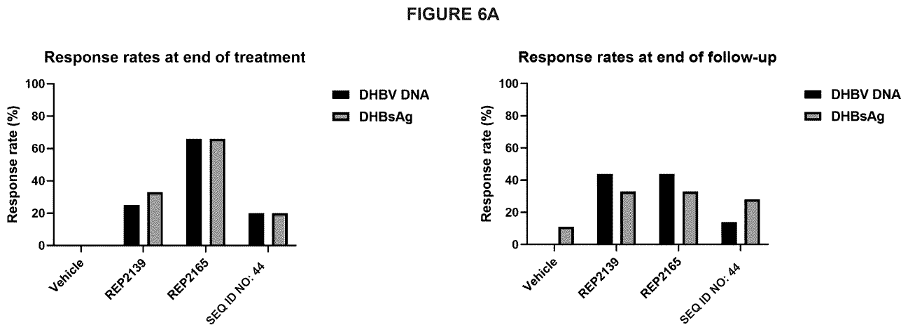

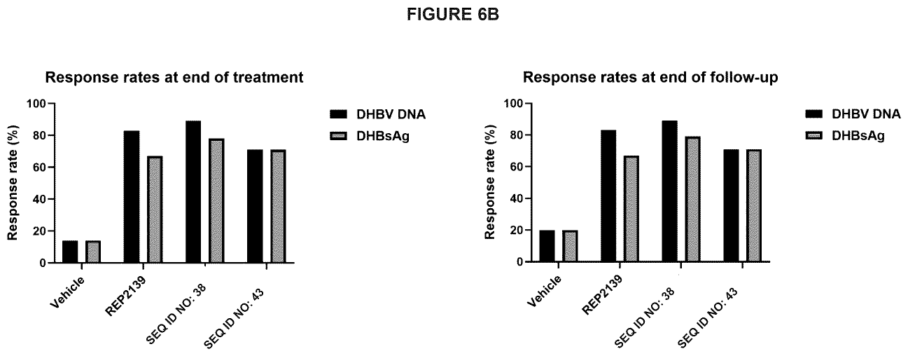

These data surprisingly showed that improved helicity in the oligonucleotides correlates with improved response. The addition of ribose units increases the homogeneity of response to treatment. Surprisingly, looking at the relative activity compared to REP-2139, treatment with compounds of the sequences of SEQ ID NO: 38 or 43 exhibited unexpected improvements over treatment with a compound of sequence SEQ ID NO: 44 (FIGS. 6A and 6B) in the DHBV duck model.

### Example 7

### Structural Differentiation of REP-2139 and SEQ ID NO: 44

The high potency of the compound with the sequence of SEQ ID NO: 44 (shown in Table 10) does not translate in clinical trials. SEQ ID NO: 44 and REP-2139 were analyzed to determine their structural differences.

TABLE 10

| SEQ ID NO: | Structure | EC₅₀ (nM) | CC₅₀ (nM) |
|---|---|---|---|
| 46 (REP2139) | All-2′-OMe | D | A |
| 44 | 50% LNA, 5 rA | A | A |
| 45 | All LNA | A | A |

A: EC₅₀ < 30 nM,
B: 30 nM ≤ EC₅₀ < 100 nM,
C: 100 nM ≤ EC₅₀ < 300 nM,
D: EC₅₀ ≥ 300 nM;
A: CC₅₀ > 500 nM and B: CC₅₀ ≤ 500 nM

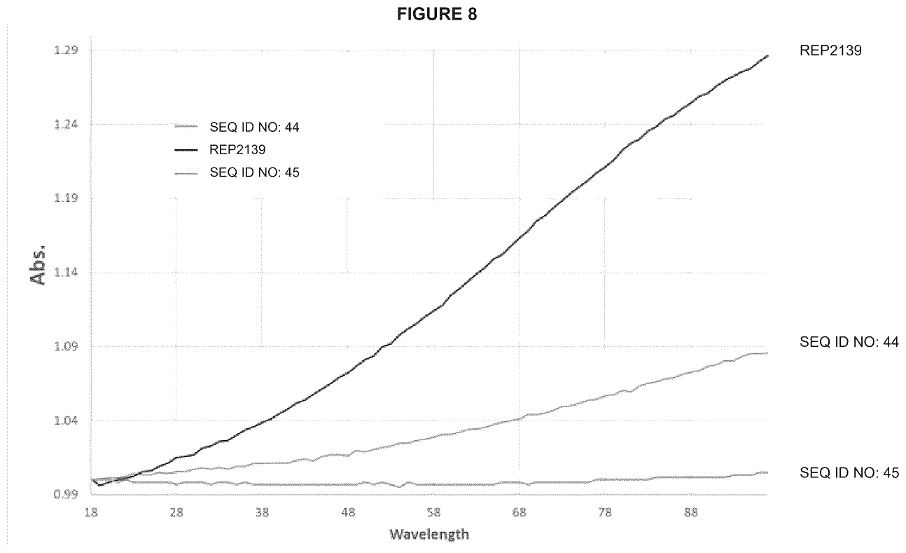

Through Tm analysis, there was a noticeable differentiation between compounds (FIG. 8). REP-2139 showed great flexibility, while SEQ ID NOs: 44 and 45 were much more rigid.

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the present disclosure.

1. A modified oligonucleotide or complex thereof having sequence independent antiviral activity against hepatitis B virus (HBV), hepatitis D virus (HDV), or both, comprising a sequence of alternating A and C units having a length of 40 units, wherein the modified oligonucleotide comprises:
i. a 5′ region comprising 8 units, wherein the A units are independently selected from 2′-OMe-A and LNA-A, and the C units are independently selected from 2′-OMe-(5m)C and LNA-(5m)C,
wherein 0, 1, 2, 3, or 4 units are independently selected from LNA-A and LNA-(5m)C;
ii. a 3′ region comprising 8 units, wherein the A units are independently selected from 2′-OMe-A, Ribo-A and LNA-A, and the C units are independently selected from 2′-OMe-(5m)C and LNA-(5m)C,
wherein 1, 2, 3 or 4 units are independently selected from LNA-A and LNA-(5m)C, and 0 or 1 of the A units is Ribo-A; and
iii. a central region comprising 24 units, wherein the A units are independently selected from 2′-OMe-A and Ribo-A, and the C units are 2′-OMe-(5m)C,
wherein 0, 1, 2, 3 or 4 of the A units are Ribo-A.

2. The modified oligonucleotide or complex thereof of claim 1, wherein all of the A units in the 5′ region and/or the 3′ region are 2′-OMe-A.

3. The modified oligonucleotide or complex thereof of claim 1, wherein all of the A units in the 5′ region are 2′-OMe-A, 3 of the A units in the 3′ region are 2′-OMe-A, and 1 of the A units in the 3′ region is Ribo-A.

4. (canceled)

5. The modified oligonucleotide or complex thereof of claim 2, wherein 1, 2, 3, or 4 of the C units of the 5′ region are LNA-(5m)C.

6. The modified oligonucleotide or complex thereof of claim 5, wherein 1, 2, or 3 of the C units of the 5′ region and/or 1, 2, or 3 units of the 3′ region are LNA-(5m)C.

7. The modified oligonucleotide or complex thereof of claim 6, wherein 2 of the C units of the 5′ region are LNA-(5m)C and/or 2 of the C units of the 3′ region are LNA-(5m)C.

8. (canceled)

9. The modified oligonucleotide or complex thereof of claim 1, wherein all of the A units of the central region are 2′-OMe-A.

10. The modified oligonucleotide or complex thereof of claim 1, wherein at least one A unit at position 11, 17, 21, 23, 29, 31, 33, 35, or any combination thereof, is a ribo-A.

11. The modified oligonucleotide or complex thereof of claim 10, wherein 1, 2, or 3 of the A units of the central region are Ribo-A.

12. The modified oligonucleotide or complex thereof of claim 11, wherein in all of the A units of the 3′ region are 2′-OMe-A.

13. The modified oligonucleotide or complex thereof of claim 12, wherein the A units at position 11, 21 and 31 are Ribo-A.

14. The modified oligonucleotide or complex thereof of claim 1, wherein the sequence is at least partially phosphorothioated.

15. (canceled)

16. The modified oligonucleotide complex of claim 1, wherein the complex is a monovalent counterion complex.

17. The modified oligonucleotide or complex thereof of claim 1, wherein the modified oligonucleotide has an EC₅₀ value against hepatitis B virus (HBV), hepatitis D virus (HDV), or both, as determined by HBsAg Secretion Assay, of less than 100 nM.

18. The modified oligonucleotide or complex thereof of claim 1, wherein the helicity of the oligonucleotide is greater than that SEQ ID NO: 44, as demonstrated by a more sigmoidal Tm curve compared with the Tm curve of SEQ ID NO: 44.

19. The modified oligonucleotide or complex thereof of claim 1, wherein the sequence of alternating A and C units is any one of the sequences of SEQ ID NO: 10-13, 15-21, 23, 25, 27-35, or 37-43.

20. The modified oligonucleotide or complex thereof of claim 1, wherein the sequence of alternating A and C units is any one of the sequences of SEQ ID NO: 38 or 43.

21. (canceled)

22. (canceled)

23. A pharmaceutical composition, comprising an amount of the modified oligonucleotide or complex thereof of claim 1; and a pharmaceutically acceptable carrier.

24. A method of treating hepatitis B, hepatitis D or both, comprising administering an effective amount of the modified oligonucleotide or complex thereof of claim 1, to a subject in need thereof.

25. (canceled)

26. (canceled)

27. (canceled)

28. (canceled)

29. The method of claim 24, further comprising administering a second treatment comprising an siRNA oligonucleotide, an anti-sense oligonucleotide, a nucleoside, an interferon, a viral entry inhibitor, an immunomodulator, a capsid assembly modulator, an anti-HBsAg mAb, or a combination thereof.

30. (canceled)

31. (canceled)

31. (canceled)
